(19) United States
(12) Patent Application Publication
(10) Pub. No.: US 2011/0177060 A1
(43) Pub. Date: Jul. 21, 2011

(54) IAP BIR DOMAIN BINDING COMPOUNDS

(75) Inventor: James Jaquith, Pincourt (CA)

(73) Assignee: AEGERA THERAPEUTICS, INC., Verdun (CA)

(21) Appl. No.: 13/119,900

(22) PCT Filed: Sep. 17, 2009
(86) PCT No.: PCT/CA09/01298

§ 371 Date: Apr. 7, 2011

Related U.S. Application Data
(60) Provisional application No. 61/098,620, filed on Sep. 19, 2008.

(51) Int. Cl. A61K 39/395; C07D 207/22; C07D 403/14; A61K 31/4025; A61K 31/506; A61K 38/16; A61P 35/00

(52) U.S. Cl. 424/130.1; 548/523; 544/296; 514/422; 514/256; 514/21.2; 514/19.3

(57) ABSTRACT

IAP BIR domain binding compounds of Formula (I), (II), (III), or (IV), and the use thereof, for example, for the treatment or prevention of proliferative diseases.

### FIELD OF THE INVENTION

The present invention concerns bridged compounds that bind to IAP BIR domains, and which are useful for treating proliferative disorders and disorders of dysregulated apoptosis, such as cancer.

### BACKGROUND OF THE INVENTION

Apoptosis, or programmed cell death, typically occurs in the normal development and maintenance of healthy tissues in multicellular organisms. It is a complex process which results in the removal of damaged, diseased or developmentally redundant cells, in the absence of signs of inflammation or necrosis.

Intrinsic apoptotic pathways are known to be dysregulated, most particularly in cancer and lymphoproliferative syndromes, as well as autoimmune disorders such as multiple sclerosis, in neurodegenerative diseases and in inflammation. As well, alterations in a host apoptotic response have been described in the development or maintenance of viral and bacterial infections.

The caspases are a family of proteolytic enzymes from the class of cysteine proteases which are known to initiate and execute apoptosis. In normal cells, the caspases are present as inactive zymogens, which are catalytically activated following external signals, for example those resulting from ligand driven Death Receptor activation, such as cytokines or immunological agents, or by release of mitochondrial factors, such as cytochrome C following genotoxic, chemotoxic, or radiation-induced cellular injury. The Inhibitors of Apoptosis Proteins (IAPs) constitute a family of proteins which are capable of binding to and inhibiting the caspases, thereby suppressing cellular apoptosis. Because of their central role in regulating Caspase activity, the IAPs are capable of inhibiting programmed cell death from a wide variety of triggers, which include loss of homeostatic, or endogenous cellular growth control mechanisms, as well as chemotherapeutic drugs and irradiation.

The IAPs contain one to three homologous structural domains known as baculovirus IAP repeat (BIR) domains. They may also contain a RING zinc finger domain at the C-terminus, with a capability of inducing ubiquitinylation of IAP-binding molecules via its E3 ligase function. The human IAPs, XIAP, HIAP1 (also referred to as cIAP2), and HIAP2 (cIAP1) each have three BIR domains, and a carboxy terminal RING zinc finger. Another IAP, NAIP, has three BIR domains (BIR1, BIR2 and BIR3), but no RING domain, whereas Livin, TsIAP and MLIAP have a single BIR domain and a RING domain. The X chromosome-linked inhibitor of apoptosis (XIAP) is an example of an IAP which can inhibit the initiator caspase, known as caspase-9, and the effector caspases, Caspase-3 and Caspase-7, by direct binding. It can also induce the removal of caspases through the ubiquitylation-mediated proteasome pathway via the E3 ligase activity of a RING zinc finger domain. It is via the BIR3 domain that XIAP binds to and inhibits caspase-9. The linker-BIR2 domain of XIAP inhibits the activity of caspases-3 and -7. The BIR domains have also been associated with the interactions of IAPs with tumor necrosis factor-receptor associated factor (TRAFs)-1 and -2, and to TAB1, as adaptor proteins effecting survival signaling through NFkB activation. The IAPs thus function as a direct brake on the apoptosis cascade, by preventing the action of, or inhibiting active caspases and by re-directing cellular signaling to a pro-survival mode.

Progress in the cancer field has led to a new paradigm in cancer biology wherein neoplasia may be viewed as a failure of cancer cells to execute normal pathways of apoptosis. Normal cells receive continuous feedback from their environment through various intracellular and extracellular factors, and “commit suicide” if removed from this context. This induction of apoptosis is achieved by activation of the caspase cascade. Cancer cells, however, gain the ability to overcome or bypass this apoptosis regulation and continue with inappropriate proliferation. The majority of treatments for cancer induce at least a partial apoptotic response in the cancer target cell, resulting in remission or initiation of tumor regression. In many cases, however, residual cells which are apoptosis-resistant are capable of escaping therapy and continuing the process of oncogenic/genetic change, resulting in the emergence of highly drug-resistant, metastatic disease which overcomes our ability to effectively treat the disease. Furthermore, most cancer therapies, including radiation therapy and traditional chemotherapy do induce apoptosis in cancer cells, but cause additional cellular injury, due to their lack of specificity in inducing apoptosis solely in cancer cells. The need to improve the specificity/potency of pro-apoptosis agents used to treat cancer, and indeed other proliferative disorders, is important because of the benefits in decreasing the side effects associated with administration of these agents. Therefore, finding novel means of inducing apoptosis in cancer cells is a highly desired medical need and its solution offers the possibility of entirely new treatments for cancer.

A growing body of data indicates that cancer cells may avoid apoptosis by the sustained over-expression of one or more members of the IAP family of proteins, as documented in many primary tumor biopsy samples, as well as most established cancer cell lines. Epidemiological studies have demonstrated that over-expression of the various IAPs is associated with poor clinical prognosis and survival. For XIAP this is shown in cancers as diverse as leukemia and ovarian cancer. Over expression of HIAP1 and HIAP2 resulting from the frequent chromosome amplification of the 11q21-q23 region, which encompasses both, has been observed in a variety of malignancies, including medulloblastomas, renal cell carcinomas, glioblastomas, and gastric carcinomas. (X)IAP negative regulatory molecules such as XAF, appear to be tumor suppressors, which are very frequently lost in clinical cancers. Thus, by their ability to suppress the activation and execution of the intrinsic mediators of apoptosis, the caspases, the IAPs may directly contribute to tumor progression and resistance to pharmaceutical intervention. Induction of apoptosis in cancer cells by the use of potent small molecules which bind to specific IAP domains is the subject of this invention.

We and others have demonstrated the critical importance of the individual BIR domains for affecting the antiapoptotic function of the IAPs. We have proposed that antagonists of the IAPs, which may bind to the individual BIR domains, would disrupt the antiapoptotic function of the IAPs. Indeed, individual BIRs serve as critical binding sites for the N-terminal Ser-Gly-Val-Asp, Ser-Gly-Pro-Ile and Ala-Thre-Pro-Ile residues of the Caspases 3, 7, and 9, respectively, and such binding is imperative for the Caspase-inhibitory function of the IAPs. The binding of N-terminal AxPy tetra-peptide residues to XIAP results in the release of the active caspases 3, 7 and 9. In the case of the other IAPs, such as c-IAP1 and c-IAP2, the functions of the BIRs, when ligand-bound, appear to direct the activation of the ubiquitin ligase RING function of the IAPs to a bound target, or individual IAPs themselves, to cause proteosomal loss. In either case, small molecule antagonists of the IAPs should be excellent pro-apoptotic agents, with potential uses in cancer, various proliferative disorders and inflammation.

A mammalian mitochondrial protein, namely Second Mitochondria-derived Activator of Caspases (SMAC) which antagonizes IAP function, binds mainly to the BIR 3 or 2 sites on respective IAPs via an AxPy amino-terminal tetrapeptide. Four Drosophila death-inducing proteins, Reaper, HID, Grim, and Sickle, which antagonize the ability of the Drosophila IAPs to inhibit caspases, also bind the BIR domains of the analogous Drosophila IAPs via a short AxPy amino-terminal tetrapeptide, a sequence that fits into the BIR binding pocket and disrupts IAP-caspase interactions.

The overall topology of individual BIR domains is highly conserved between the human IAPs and between individual BIR domains of the human IAPs, each BIR being a zinc finger polypeptide domain, locked into a coordinated Zn atom by two cysteines and a histidine residue. The X-ray crystallographic structures of XIAP BIR2 and BIR3 reveal a critical binding pocket for an AXPY motif on the surface of each BIR domain. There are alterations in the intervening amino acid sequences that form the binding pocket and groove in both BIR2 and BIR3. Likewise, we have described homologous domains in the BIRs of other IAPs clAP1 and clAP2. This opens the possibility of obtaining various classes of natural and synthetic binding compounds which will have different specificity and binding affinities between each of the BIR domains for each of the IAPs. Discerning the way in which such compounds will affect the biological function of the IAPs in cancer cells vs normal cells is a major new challenge in the discovery of novel mechanism agents to treat cancer and other proliferative disorders where dysregulated IAP function is observed. It is our finding that certain classes of BIR binding compounds may bind to IAP BIRs, with unexpected selectivity and potency, resulting in distinct therapeutic advantages for certain structural classes, potentially resulting from either IAP loss of function or loss of cellular IAP protein, or both.

A number of peptidic AxPy-like and heterocyclic modified AxPy peptidic compounds have been described which activate cellular Caspase 3 by reportedly binding to XIAP BIR3. For recent reviews, see Elmore et al., Annual Reports in Medicinal Chemistry, 40 (2006) 245-262; Sun et al., Bioorg. Med. Chem. Let. 15 (2005) 793-797; Oost et al., J. Med. Chem., 2004, 47(18), 4417-4426; Park et al., Bioorg. Med. Chem. Lett. 15 (2005) 771-775; Franklin et al., Biochemistry, Vol. 42, No. 27, 2003, 8223-8231; Kip et al., Biochemistry 2002, 41, 7344-7349; Wu et al., Chemistry and Biology, Vol. 10, 759-767 (2003); Glover et al., Analytical Biochemistry, 320 (2003) 157-169; United States published patent application number 20020177557; United States published patent application number 20040180828; United States published patent application number US2006/0025347A1; United States published patent application number US2005/0197403A1; and United States published patent application number US2006/0194741A1.

The aforesaid compounds have been shown to target an isolated BIR3 domain of XIAP via displacement of a fluorescently-labeled probe and they appear to induce an apoptotic event in a select set of cancer cell lines with potency in the low micromolar-nanomolar range. These compounds displayed poor in-vivo activity, likely due to limited bioavailability and may therefore have limited therapeutic application.

Thus, IAP BIR domains represent an attractive target for the discovery and development of novel therapeutic agents, especially for the treatment of proliferative disorders such as cancer.

### SUMMARY OF THE INVENTION

In one embodiment of the present invention, there is provided a compound represented by Formula I:

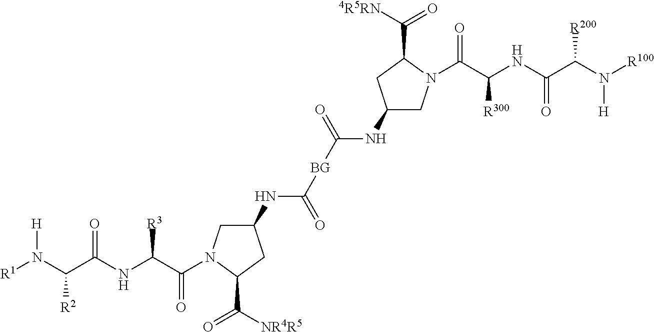

or a salt thereof, wherein:
BG is cyclohexyl, phenyl, naphthyl or biphenyl optionally substituted with R¹⁰;
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents;
R⁴ is H or C₁-C₆ alkyl;
R⁵ is

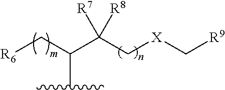

wherein:
m is 0 or 1;
n is 0 or 1;
X is O, S, or SO₂;
R⁶ is C₆-C₁₀ aryl optionally substituted with one or more R¹¹ substituents;
R⁷ and R⁸ are both hydrogen or taken together form (═O);
R⁹ is
   1) C₁-C₁₀ alkyl optionally substituted with one or more R¹⁰ substituents;
   2) naphthyl optionally substituted with one or more R¹⁰ substituents; or
   3) phenyl substituted with one or more R¹⁰ substituents;
R¹⁰ is C₁-C₆ alkyl or C₆-C₁₀ aryl optionally substituted with C₁-C₆ alkyl;
R¹¹ is
   1) halogen,
   2) OR¹²,
   3) SR¹²,
   4) NO₂,
   5) CN,
   6) haloalkyl,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl, or
   12) C₆-C₁₀ aryl;
and R¹² is C₁-C₆ alkyl.

In another embodiment, the invention provides a compound represented by Formula I:

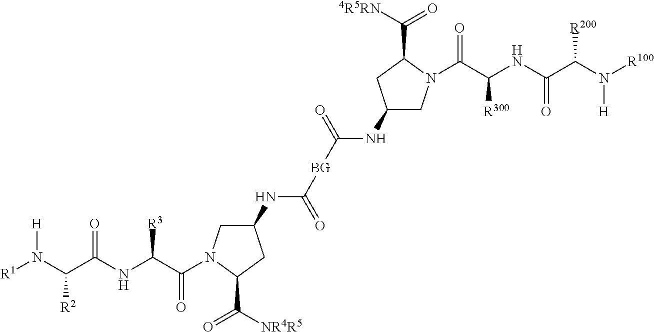

or a salt thereof, wherein:
BG is cyclohexyl, phenyl, naphthyl or biphenyl optionally substituted with R¹⁰;
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents;
R⁴ is H or C₁-C₆ alkyl;
R⁵ is

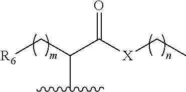

wherein:
m is 0 or 1;
n is 0-6;
X is O or S;
R⁶ is C₆-C₁₀ aryl optionally substituted with one or more R¹¹ substituents;
R¹⁰ is C₁-C₆ alkyl or C₆-C₁₀ aryl optionally substituted with C₁-C₆ alkyl;
R¹¹ is
   1) halogen,
   2) OR¹²,
   3) SR¹²,
   4) NO₂,
   5) CN,
   6) haloalkyl,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl, or
   12) C₆-C₁₀ aryl;
and R¹² is C₁-C₆ alkyl.

In another embodiment, the invention provides a compound represented by Formula II:

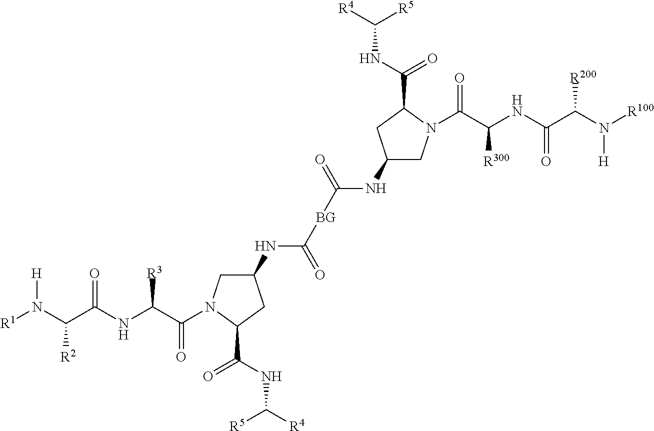

or a salt thereof, wherein:
BG is cyclohexyl, phenyl, naphthyl or biphenyl optionally substituted with R¹⁰
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents;
R⁴ is C₆-C₁₀ aryl substituted with R¹¹;
R⁵ is C₆-C₁₀ aryl optionally substituted with R¹¹, wherein R⁴ and R⁵ are not the same;
R¹⁰ is C₁-C₆ alkyl or C₆-C₁₀ aryl optionally substituted with C₁-C₆ alkyl;
R¹¹ is
   1) halogen,
   2) OR¹²,
   3) SR¹²,
   4) NO₂,
   5) CN,
   6) haloalkyl,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl, or
   12) C₆-C₁₀ aryl;
and R¹² is C₁-C₆ alkyl.

In another embodiment, the invention provides a compound represented by Formula III:

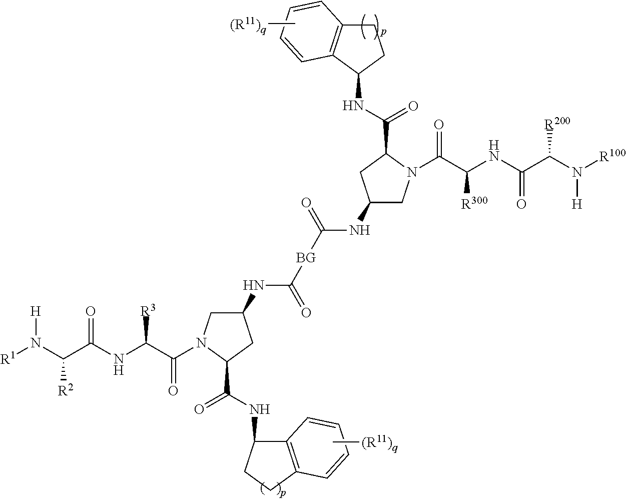

or a salt thereof, wherein:
BG is cyclohexyl, phenyl, naphthyl or biphenyl optionally substituted with R¹⁰;
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents;
p is 1 or 3;
q is 0 to 4;
R¹⁰ is C₁-C₆ alkyl or C₆-C₁₀ aryl optionally substituted with C₁-C₆ alkyl;
R¹¹ is
   1) halogen,
   2) OR¹²,
   3) SR¹²,
   4) NO₂,
   5) CN,
   6) haloalkyl,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl, or
   12) C₆-C₁₀ aryl;
and R¹² is C₁-C₆ alkyl.

In another embodiment, the invention provides a compound represented by Formula IV:

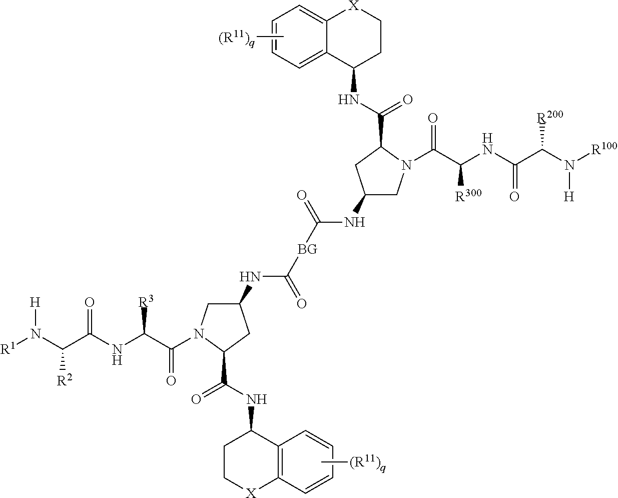

or a salt thereof, wherein:
BG is cyclohexyl, phenyl, naphthyl or biphenyl optionally substituted with R¹⁰;
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents;
X is O, S, or SO₂;
q is 0 to 4;
R¹⁰ is C₁-C₆ alkyl or C₆-C₁₀ aryl optionally substituted with C₁-C₆ alkyl;
R¹¹ is
   1) halogen,
   2) OR¹²,
   3) SR¹²,
   4) NO₂,
   5) CN,
   6) haloalkyl,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl, or
   12) C₆-C₁₀ aryl;
and R¹² is C₁-C₆ alkyl.

In another embodiment, the invention provides a compound represented by Formula V

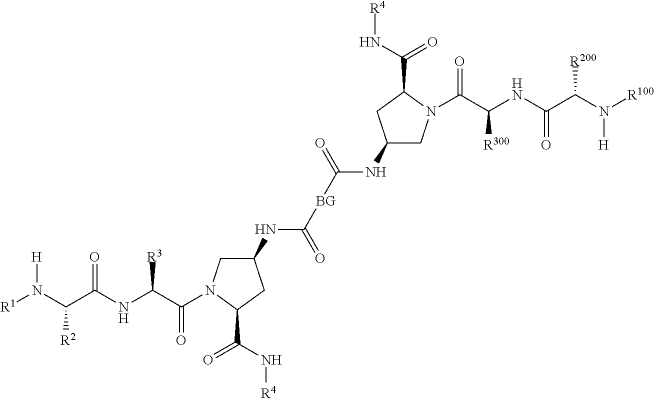

or a salt thereof, wherein:
BG is cyclohexyl, phenyl, naphthyl or biphenyl optionally substituted with R¹⁰;
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents;
X is O, S, or SO₂;
q is 0 to 4;
R⁴ is aryl or heteroaryl which is further substituted with a heteroaryl;
R¹⁰ is C₁-C₆ alkyl or C₆-C₁₀ aryl optionally substituted with C₁-C₆ alkyl;
R¹¹ is
   1) halogen,
   2) OR¹²,
   3) SR¹²,
   4) NO₂,
   5) CN,
   6) haloalkyl,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl, or
   12) C₆-C₁₀ aryl;
and R¹² is C₁-C₆ alkyl.

In another aspect of the present invention, there is provided a pharmaceutical composition comprising a compound, as described above, and a carrier, diluent or excipient, optionally in combination with one or more additional active compounds.

In another aspect of the present invention, there is provided a method of treating a disease state characterized by insufficient apoptosis, the method comprising: administering to a subject in need thereof, a therapeutically effective amount of a compound or pharmaceutical composition, as described above, so as to treat the disease state.

In another aspect of the present invention, there is provided a method of modulating IAP function, the method comprising: contacting a cell with a compound of the present invention so as to prevent binding of a BIR binding protein to an IAP BIR domain thereby modulating the IAP function.

In another aspect of the present invention, there is provided a method of treating a proliferative disease, the method comprising: administering to a subject in need thereof, a therapeutically effective amount of a compound or pharmaceutical composition, as described above, so as to treat the proliferative disease.

In another aspect of the present invention, there is provided a method of treating cancer, the method comprising: administering to a subject in need thereof, a therapeutically effective amount of a compound or pharmaceutical composition, as described above, so as to treat the cancer.

In another aspect of the present invention, there is provided a method of treating cancer, the method comprising: administering to the subject in need thereof, a therapeutically effective amount of a compound or pharmaceutical composition, as described above, in combination or sequentially with an agent selected from:
a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-γ agonist,
m) a PPAR-δ agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug,
s) a proteasome inhibitor,
t) an HDAC inhibitor,
u) an inhibitor of the chemotrypsin-like activity in the proteasome, or
v) E3 ligase inhibitors,
w) a modulator of the immune system such as, but not limited to, interferon-alpha, Bacillus Calmette-Guerin (BCG), and ionizing radition (UVB) that can induce the release of cytokines, such as the interleukins, TNF, or induce release of death receptor ligands such as TRAIL,
x) a modulator of TRAIL death receptors and TRAIL receptor agonists such as the humanized antibodies HGS-ETR1 and HGS-ETR2,
   or in combination or sequentially with radiation therapy, so as to treat the cancer.

In another aspect of the present invention, there is provided a method for the treatment or prevention of a proliferative disorder in a subject, the method comprising administering to the subject a therapeutically effective amount of a compound or pharmaceutical composition, described above.

In another aspect of the present invention, the method further comprises administering to the subject a therapeutically effective amount of a chemotherapeutic agent prior to, simultaneously with or after administration of the compound or pharmaceutical composition.

In yet another aspect, the method further comprises administering to the subject a therapeutically effective amount of a death receptor agonist prior to, simultaneously with or after administration of the compound or pharmaceutical composition. The death receptor agonist is TRAIL or the death receptor agonist is a TRAIL receptor antibody. The death receptor agonist is typically administered in an amount that produces a synergistic effect.

In yet another aspect, there is provided use of the compound as described above for the manufacture of a medicament for treating or preventing a disease state characterized by insufficient apoptosis.

In yet another aspect, there is provided use of the compound as described above for the manufacture of a medicament for treating or preventing a proliferative disorder.

In yet another aspect, there is provided use of the compound as described above in combination with an agent for the manufacture of a medicament for treating or preventing a proliferative disorder, wherein the agent is selected from:
a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-.γ agonist,
m) a PPAR-.δ agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug,
s) a proteasome inhibitor,
t) an HDAC inhibitor,
u) an inhibitor of the chemotrypsin-like activity in the proteasome, or
v) E3 ligase inhibitors,
w) a modulator of the immune system such as, but not limited to, interferon-alpha, Bacillus Calmette-Guerin (BCG), and ionizing radition (UVB) that can induce the release of cytokines, such as the interleukins, TNF, or induce release of death receptor ligands such as TRAIL,
x) a modulator of TRAIL death receptors and TRAIL receptor agonists such as the humanized antibodies HGS-ETR1 and HGS-ETR2,
   or in combination or sequentially with radiation therapy.

In yet another aspect, there is provided use of the compound as described above in combination with a death receptor agonist for the manufacture of a medicament for the treatment or prevention of a proliferative disorder in a subject.

In yet another aspect, there is provided a pharmaceutical composition comprising the compound as described above, mixed with a pharmaceutically acceptable carrier, diluent or excipient, for treating or preventing a disease state characterized by insufficient apoptosis.

In yet another aspect, there is provided a pharmaceutical composition comprising the compound as described above in combination with any compound that increases the circulating level of one or more death receptor agonists for preventing or treating a proliferative disorder.

In another aspect of the present invention, there is provided a probe, the probe being a compound of Formula I, II, III, or IV above, the compound being labeled with a detectable label or an affinity tag.

In another aspect of the present invention, there is provided a method of identifying compounds that bind to an IAP BIR domain, the assay comprising:
a) contacting an IAP BIR domain with a probe to form a probe:BIR domain complex, the probe being displaceable by a test compound,
b) measuring a signal from the probe so as to establish a reference level,
c) incubating the probe:BIR domain complex with the test compound,
d) measuring the signal from the probe,
e) comparing the signal from step d) with the reference level, a modulation of the
signal being an indication that the test compound binds to the BIR domain,
   wherein the probe is a compound of Formula I, II, III, or IV labeled with a detectable label or an affinity label.

In another aspect of the present invention, there is provided a method of detecting loss of function or suppression of IAPs in vivo, the method comprising: a) administering to a subject, a therapeutically effective amount of a pharmaceutical composition, as defined above; b) isolating a tissue sample from the subject; and c) detecting a loss of function or suppression of IAPs from the sample.

### DETAILED DESCRIPTION OF THE INVENTION

The invention provides, in several embodiments, compounds of Formula I, II, III, or IV, as described herein. In accordance with any of the foregoing embodiments, R¹ and R¹⁰⁰ can be, independently, H or C₁-C₆ alkyl optionally substituted with one or more R¹¹ substituents. Desirably, R¹ and R¹⁰⁰ are C₁-C₆ alkyl or C₁-C₃, such as methyl or ethyl.

Also with respect to any of the foregoing embodiments, R² and R²⁰⁰ are, independently, C₁-C₆ alkyl optionally subtituted with one or more halogens, such as a C₁-C₃ alkyl optionally subtituted with one or more halogens. Desirably, R² and R²⁰⁰ are both methyl or ethyl.

Similarly, R³ and R³⁰⁰ in any of the foregoing embodiments are, independently, C₁-C₆ alkyl optionally subtituted with one or more R¹¹ substituents. For example, R³ and R³⁰⁰ can each be a C¹-C⁴ alkyl, such as C(CH₃)₃.
R¹¹ is
   1) halogen,
   2) OR¹²,
   3) SR¹²,
   4) NO₂,
   5) CN,
   6) haloalkyl,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl, or
   12) C₆-C₁₀ aryl;
      and R¹² is C₁-C₆ alkyl.

BG in any of the foregoing embodiments can be cyclohexyl, phenyl, naphthyl or biphenyl optionally substituted with R¹⁰, wherein In a subset of compounds, BG is phenyl.

In one embodiment of the present invention, there is provided a compound represented by Formula I:

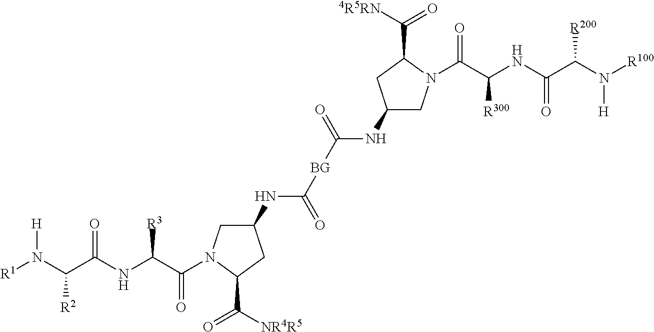

or a salt thereof, wherein R⁴ is H or C₆-C₁₀ aryl substituted with R¹¹; and R⁵ is

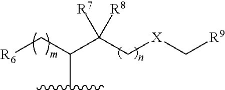

wherein m is 0 or 1; n is 0 or 1; and X is O, S, or SO₂, preferably O or S.

In accordance with this embodiment, R⁶ can be any C₆-C₁₀ aryl optionally substituted with one or more R¹¹ substituents. For example, R⁶ can be a phenyl, indanyl, 1-naphthyl, 2-naphthyl, or tetrahydronaphthyl. According to more specific embodiments, R⁶ is phenyl.

In accordance with the foregoing embodiment, R⁷ and R⁸ can both be hydrogen or can be taken together to form (═O). In a subset of compounds according to formula I, R⁷ and R⁸ are hydrogen.

R⁹ can be 1) C₁-C₁₀ alkyl optionally substituted with one or more R¹⁰ substituents; 2) naphthyl optionally substituted with one or more R¹⁰ substituents; or 3) phenyl substituted with one or more R¹⁰ substituents; wherein R¹⁰ is C₁-C₆ alkyl or C₆-C₁₀ aryl optionally substituted with C₁-C₆ alkyl. By way of illustration, R⁹ can be C₄-C₈ alkyl, which can be branched or unbranched, substituted or unsubstituted, or R⁹ can be C₁-C₃ alkyl substituted with an aryl, such as phenyl, which can be substituted with C₁-C₃ alkyl. Alternatively, R⁹ can be 1-naphthyl or 2-naphthyl, or R⁹ can be a phenyl substituted with a C₁-C₆ alkyl.

More specific examples of R⁵ in accordance with this embodiment of the invention include, without limitation:

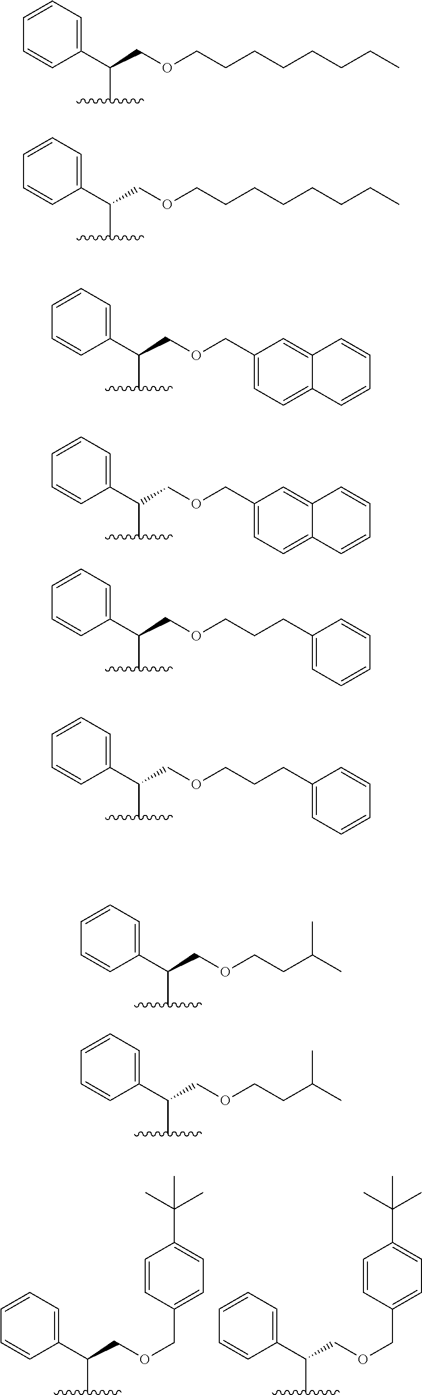

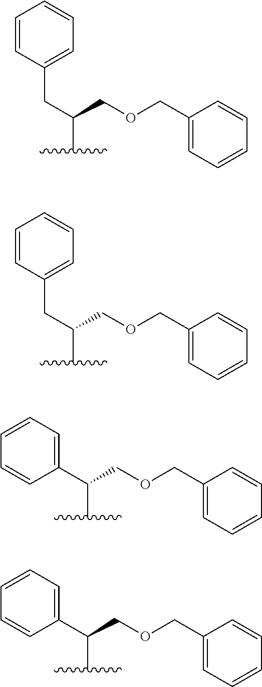

By way of further illustration, specific examples of compounds in accordance with this embodiment include compounds 1-7, as follows:

| Cmpd. | Structure |
|---|---|
| 1 | 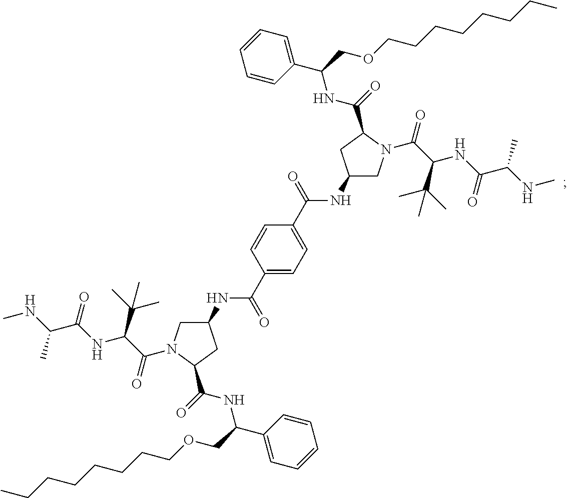 |
| 2 | 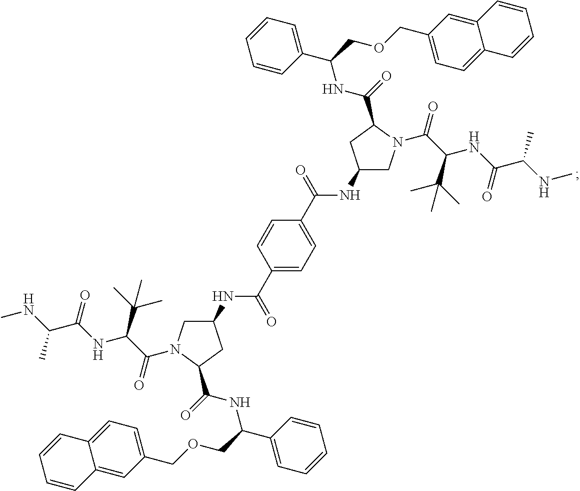 |
| 3 | 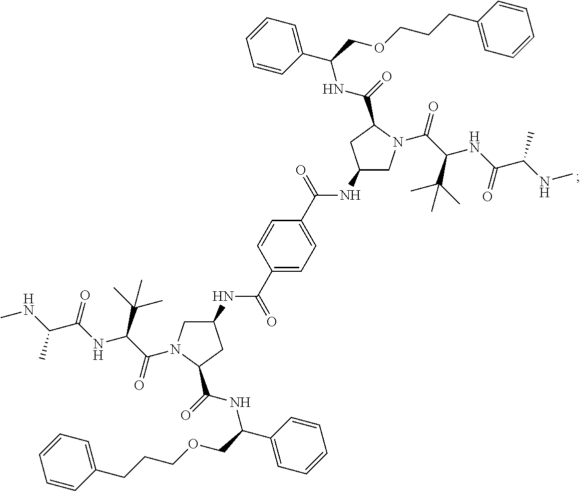 |
| 4 | 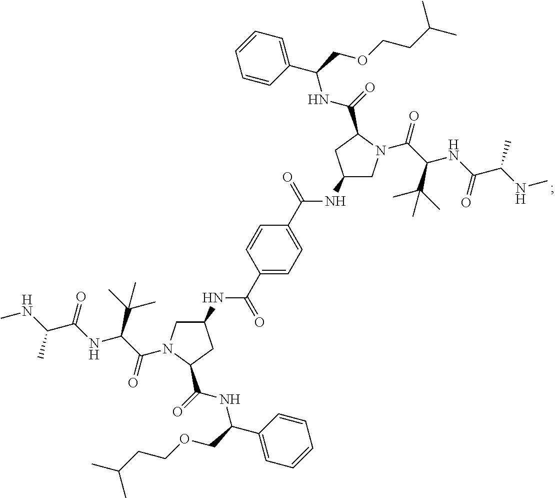 |
| 5 | 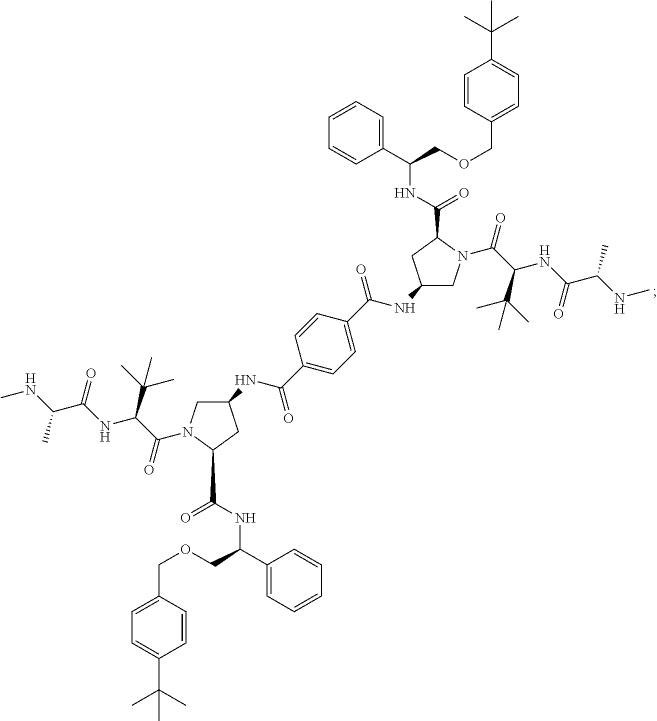 |
| 6 | 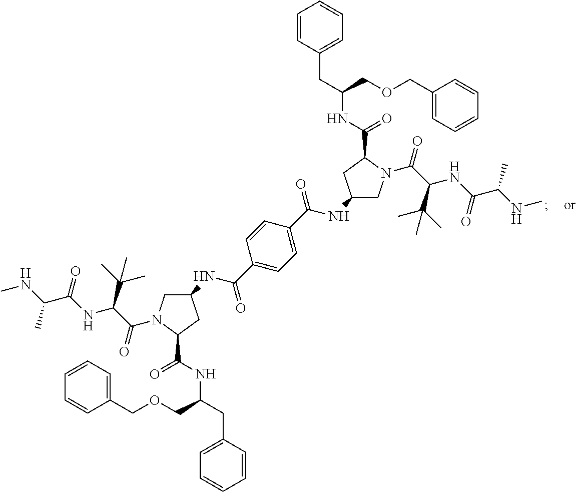 |
| 7 | 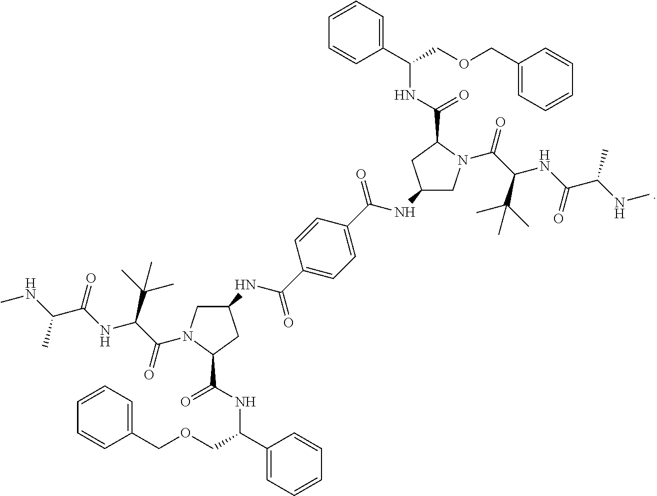 |

In another embodiment, the invention provides a compound represented by Formula I:

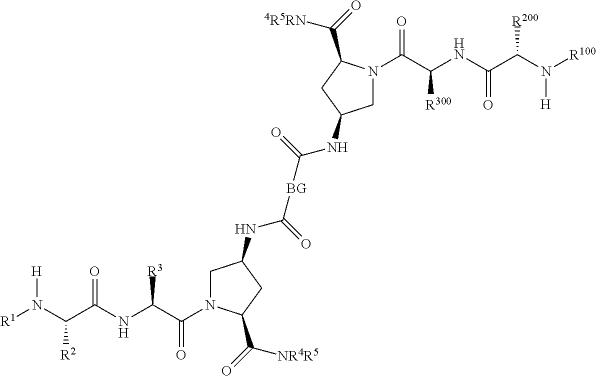

or a salt thereof, wherein R⁴ is H or C₁-C₆ alkyl substituted with R¹¹; R⁵ is

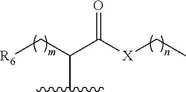

m is 0 or 1; n is 0-6; and X is O or S, preferably O.

In accordance with this embodiment, R⁶ can be any C₆-C₁₀ aryl optionally substituted with one or more R¹¹ substituents. For example, R⁶ can be a phenyl, indanyl, 1-naphthyl, 2-naphthyl, or tetrahydronaphthyl. According to more specific embodiments, R⁶ is phenyl. In addition, or alternatively, m can be 0 and n can be 0 to 3. In accordance with this embodiment, non-limiting examples of R⁵ include:

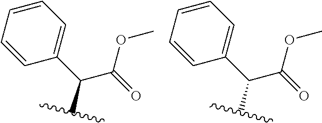

By way of further illustration, a specific example of a compound in accordance with this embodiment includes:

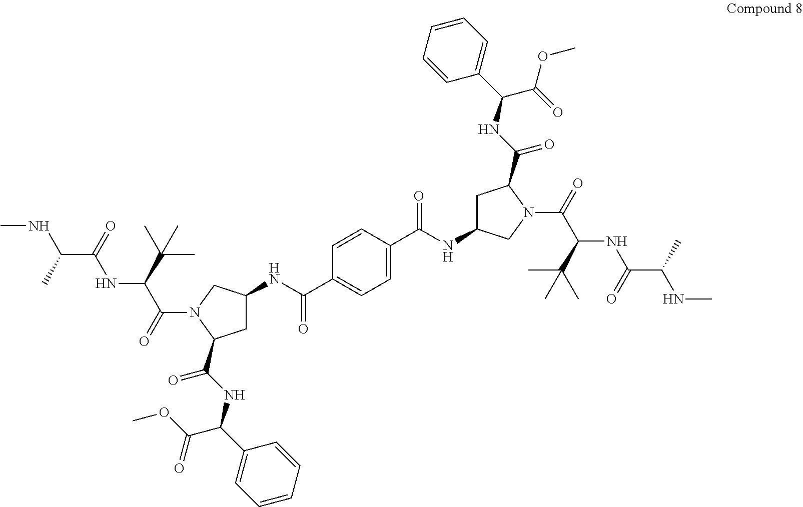

In another embodiment, the invention provides a compound represented by Formula II:

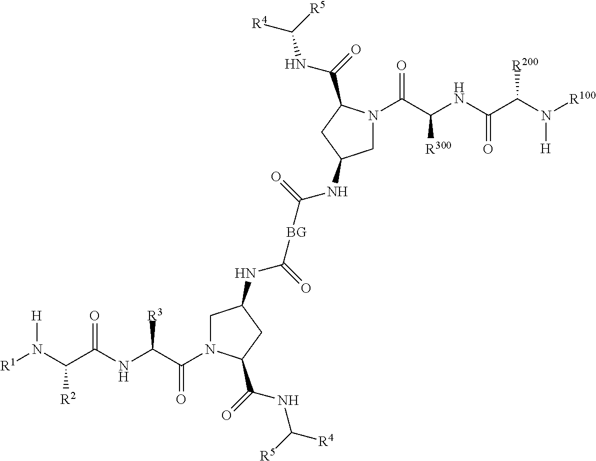

or a salt thereof, wherein R⁴ is C₆-C₁₀ aryl substituted with R¹¹, R⁵ is C₆-C₁₀ aryl optionally substituted with R¹¹, and R⁴ and R⁵ are not the same. R⁴ and R⁵ can be any aryl, including phenyl, indanyl, 1-naphthyl, 2-naphthyl, or tetrahydronaphthyl. In certain subsets of the compounds of Formula II, R⁴ and R⁵ are both phenyl, wherein R⁵ is unsubstituted and R⁴ is substituted with a C₁-C₄ alkyl or OR¹², and R¹² is a C₁-C₆ alkyl or C₁-C₃ alkyl.

By way of further illustration, specific examples of compounds in accordance with this embodiment include:

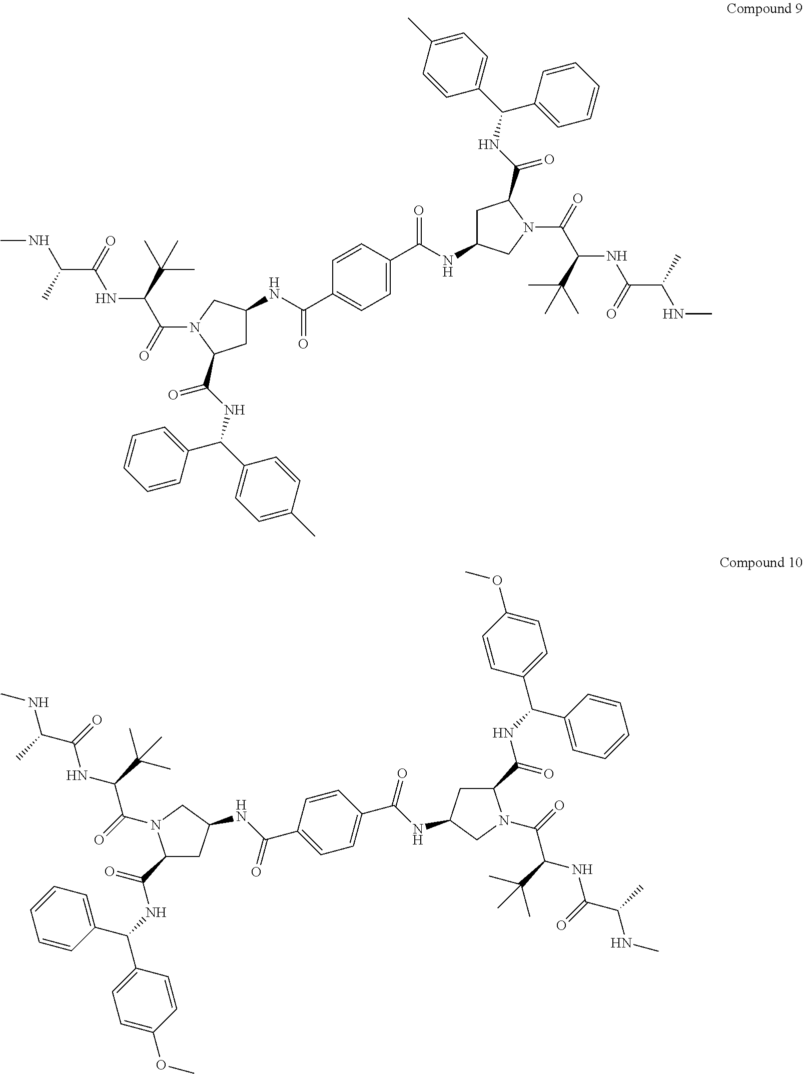

In another embodiment, the invention provides a compound represented by Formula III:

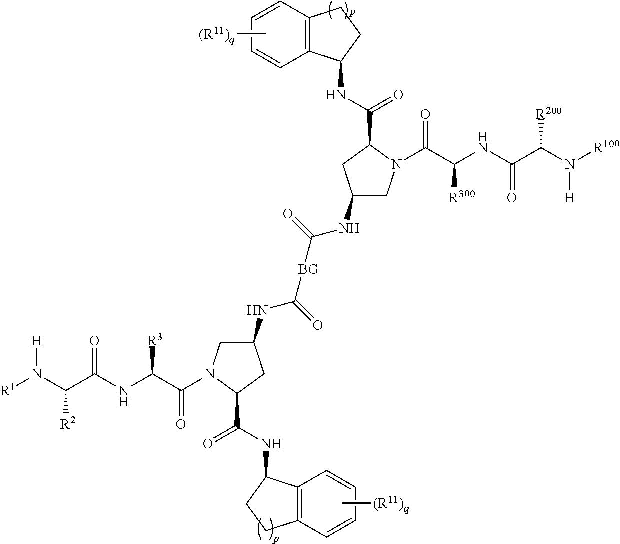

or a salt thereof, wherein p is 1 or 3 and q is 0 to 4, such as 0 to 2. In certain embodiments, q is 0. By way of further illustration, specific examples of compounds in accordance with this embodiment include:

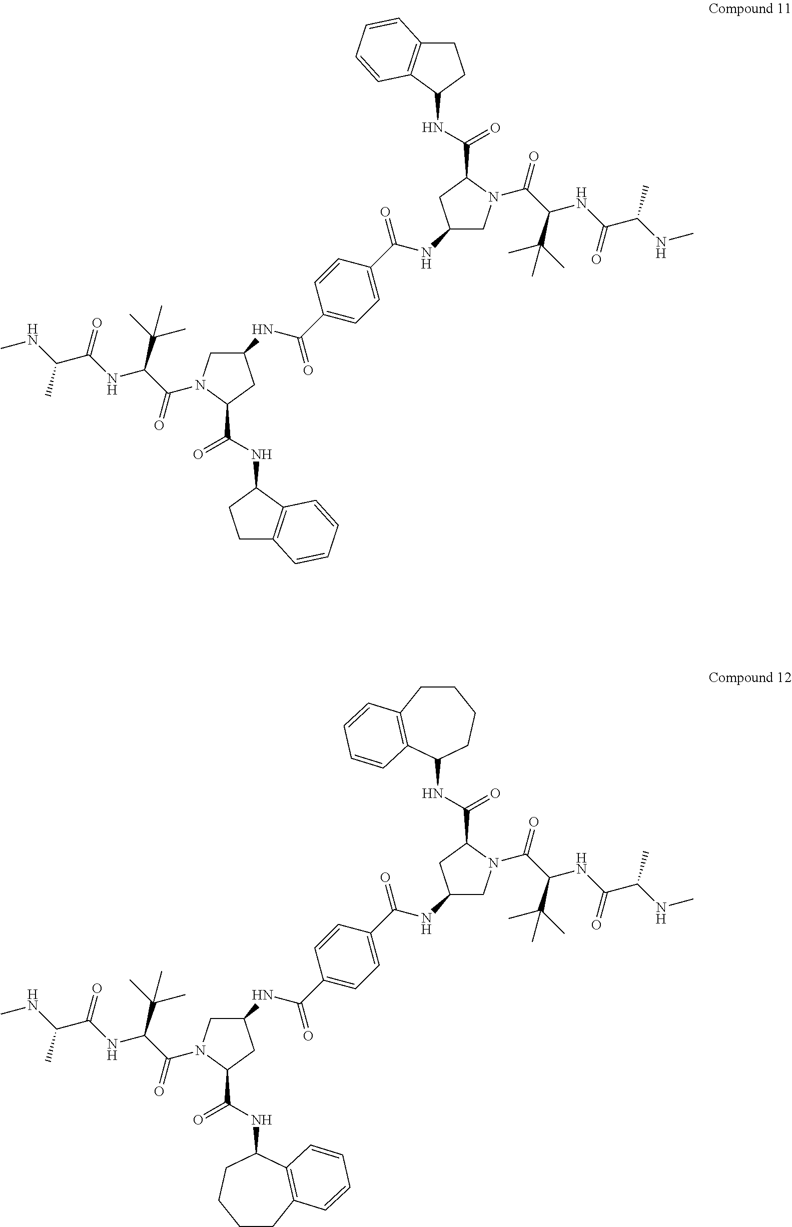

In another embodiment, the invention provides a compound represented by Formula IV:

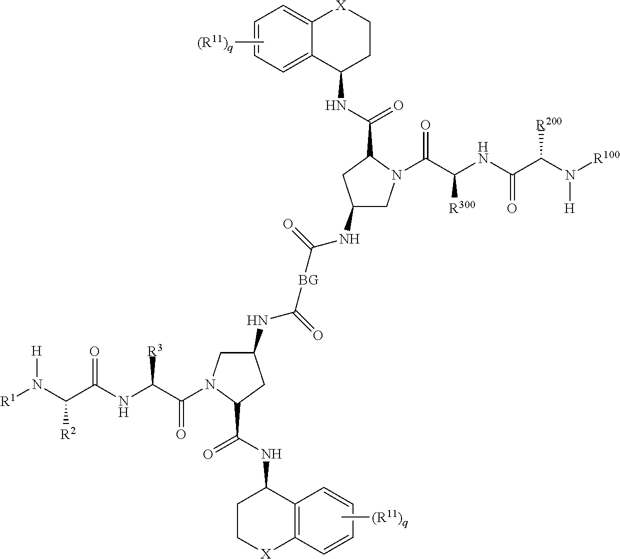

or a salt thereof, wherein X is O, S, or SO₂; and q is 0 to 4 such as 0 to 2. In certain embodiments, q is 0. By way of further illustration, specific examples of compounds in accordance with this embodiment include:

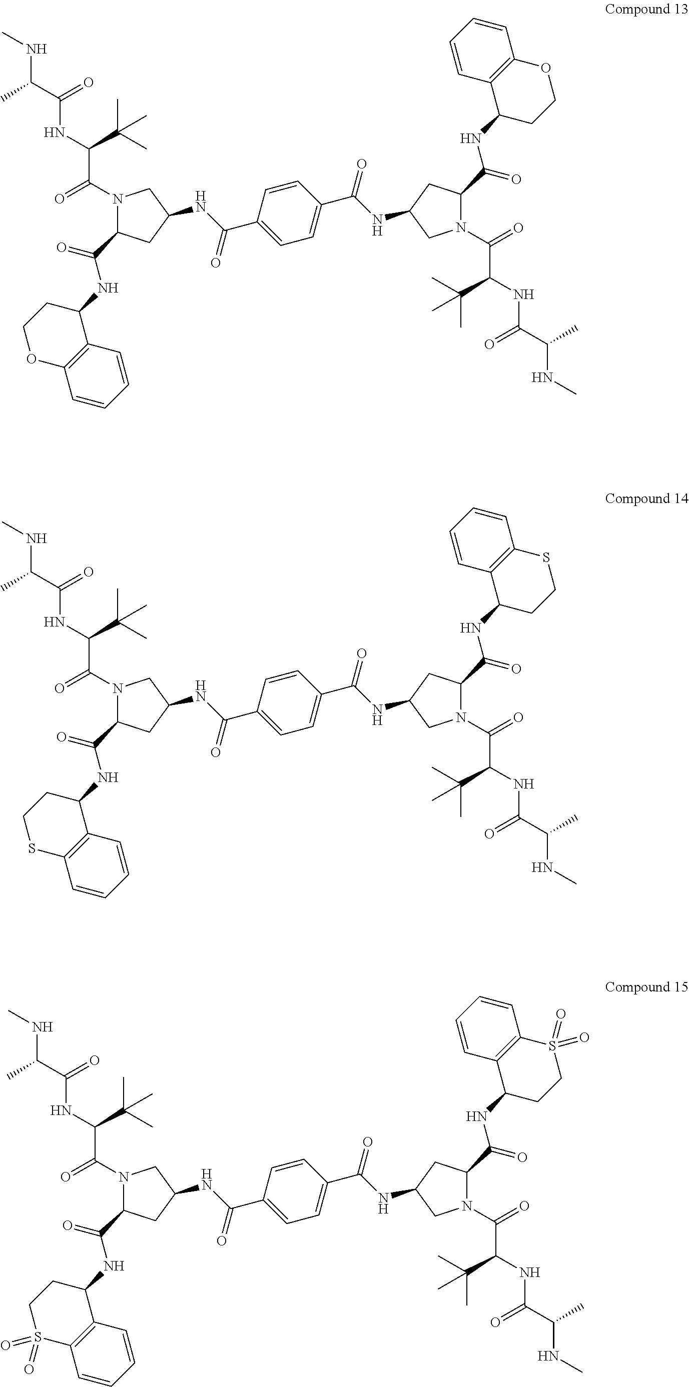

In another embodiment, the invention provides a compound represented by Formula V

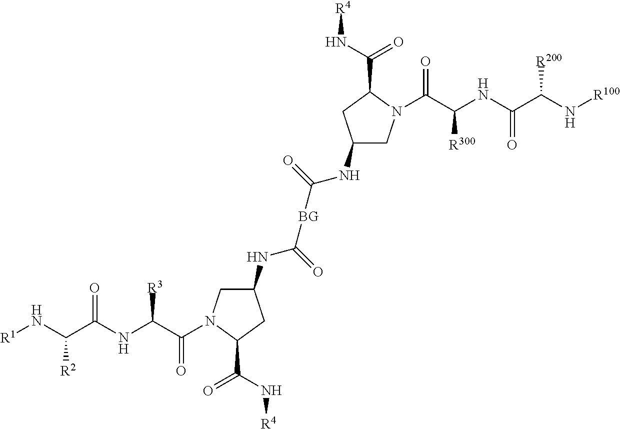

wherein R⁴ is aryl or heteroaryl, which is further substituted with a heteroaryl. An example of R⁴ is illustrated below:

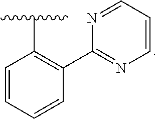

By way of further illustration, specific examples of compounds in accordance with this embodiment include:

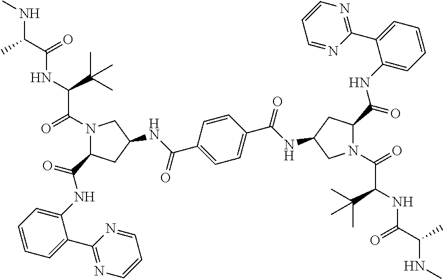

One skilled in the art will understand that substitution patterns and substituents on compounds of the present invention may be selected to provide compounds that are chemically stable and can be readily synthesized using the chemistry set forth in the examples and chemistry techniques well known in the art using readily available starting materials.

Furthermore, any of the foregoing compounds can be provided as a salt, especially a pharmaceutical salt.

Definitions

Unless otherwise specified, the following definitions apply:

As used herein, the term “alkyl” is intended to include both branched and straight chain saturated aliphatic hydrocarbon groups having the specified number of carbon atoms, for example, C₁-C₆ as in C₁-C₆-alkyl is defined as including groups having 1, 2, 3, 4, 5 or 6 carbons in a linear or branched arrangement, and C₁-C₄ as in C₁-C₄ alkyl is defined as including groups having 1, 2, 3, or 4 carbons in a linear or branched arrangement, and for example, C₁-C₂₀ as in C₁-C₂₀-alkyl is defined as including groups having 1, 2, 3, 4, 5, 6, 7, 8, 9, 10, 11, 12, 13, 14, 15, 16, 17, 18, 19 or 20 carbons in a linear or branched arrangement, Examples of C₁-C₆-alkyl and C₁-C₄ alkyl as defined above include, but are not limited to, methyl, ethyl, n-propyl, i-propyl, n-butyl, t-butyl, i-butyl, pentyl and hexyl.

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regiochemistry and combinations thereof. For example, C₂-C₆ as in C₂-C₆ alkenyl is defined as including groups having 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, 1-butenyl and the like.

As used herein, the term “alkynyl” is intended to mean unsaturated, straight chain hydrocarbon groups having the specified number of carbon atoms therein and in which at least two carbon atoms are bonded together by a triple bond. For example C₂-C₄ as in C₂-C₄ alkynyl is defined as including groups having 2, 3, or 4 carbon atoms in a chain, at least two of the carbon atoms being bonded together by a triple bond. Examples of such alkynyls include ethynyl, 1-propynyl, 2-propynyl and the like.

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3, 4, 5, 6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl.

As used herein, the term “cycloalkenyl” is intended to mean a monocyclic saturated aliphatic hydrocarbon group having the specified number of carbon atoms therein, for example, C₃-C₇ as in C₃-C₇ cycloalkenyl is defined as including groups having 3, 4, 5, 6, or 7 carbons in a monocyclic arrangement. Examples of C₃-C₇ cycloalkenyl as defined above include, but are not limited to, cyclopentenyl, and cyclohexenyl.

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “aryl”, either alone or in combination with another radical, means a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, 1-naphthyl, 2-naphthyl and tetrahydronaphthyl. The aryls may be connected to another group either at a suitable position on the cycloalkyl ring or the aromatic ring. For example:

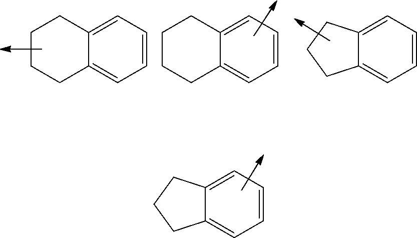

Arrowed lines drawn from the ring system indicate that the bond may be attached to any of the suitable ring atoms.

As used herein, the term “biphenyl” is intended to mean two phenyl groups bonded together at any one of the available sites on the phenyl ring. For example:

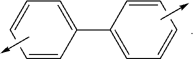

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, and

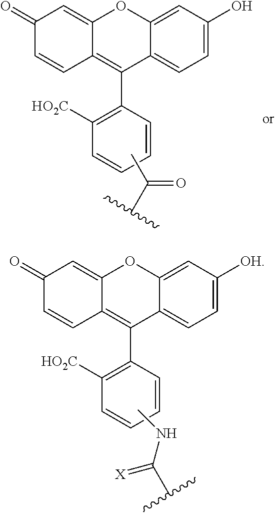

fluoroscein derivatives such as:

As used herein, the term “heterocycle”, “heterocyclic” or “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl, imidazolidinyl, morpholinyl, imidazolinyl, pyrazolidinyl, pyrazolinyl, and

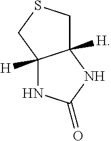

As used herein, the term “heterobicycle” either alone or in combination with another radical, is intended to mean a heterocycle as defined above fused to another cycle, be it a heterocycle, an aryl or any other cycle defined herein. Examples of such heterobicycles include, but are not limited to, coumarin, benzo[d][1,3]dioxole, 2,3-dihydrobenzo[b][1,4]dioxine and 3,4-dihydro-2H-benzo[b][1,4]dioxepine.

As used herein, the term “heterocycle”, “heterocyclic” or “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl, imidazolidinyl, morpholinyl, imidazolinyl, pyrazolidinyl, and pyrazolinyl.

As used herein, the term “heteroatom” is intended to mean O, S or N.

As used herein, the term “activated diacid” is intended to mean a diacid wherein the carboxylic acid moieties have been transformed to, for example, but not limited to, acid halides, succinate esters, or HOBt esters, either in situ or in a separate synthetic step. For example, succinyl chloride and terephthaloyl chloride are examples of “diacid chlorides.” HOBt esters can be formed in situ by the treatment of a diacid with a dehydrating agent such as DCC, EDC, HBTU, or others, a base such as DIPEA, and HOBt in an appropriate solvent. The reaction of an activated diacid with an amine will result in the conversion of the acid functionality to an amide functionality.

As used herein, the term “detectable label” is intended to mean a group that may be linked to a compound of the present invention to produce a probe or to an IAP BIR domain, such that when the probe is associated with the BIR domain, the label allows either direct or indirect recognition of the probe so that it may be detected, measured and quantified.

As used herein, the term “affinity tag” is intended to mean a ligand or group, which is linked to either a compound of the present invention or to an IAP BIR domain to allow another compound to be extracted from a solution to which the ligand or group is attached.

As used herein, the term “probe” is intended to mean a compound described herein which is labeled with either a detectable label or an affinity tag, and which is capable of binding, either covalently or non-covalently, to an IAP BIR domain. When, for example, the probe is non-covalently bound, it may be displaced by a test compound. When, for example, the probe is bound covalently, it may be used to form cross-linked adducts, which may be quantified and inhibited by a test compound.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not.

If the substituents themselves are incompatible with the synthetic methods of the present invention, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety. Examples of protecting groups used throughout include, but are not limited to Fmoc, Bn, Boc, CBz and COCF₃. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

Abbreviations for α-amino acids used throughout are as follows:

| Amino acid | Abbreviation |
|---|---|
| α-Amino butyric acid | Abu |
| Alanine | Ala |
| Arginine | Arg |
| Aspartic acid | Asp |
| Asparagine | Asn |
| Cysteine | Cys |
| Glutamic acid | Glu |
| Glutamine | Gln |
| Glycine | Gly |
| Isoleucine | Ile |
| Histidine | His |
| Leucine | Leu |
| Lysine | Lys |
| Methionine | Met |
| Phenylalanine | Phe |
| Proline | Pro |
| Serine | Ser |
| Threonine | Thr |
| Tryptophan | Trp |
| Tyrosine | Tyr |
| Valine | Val |

As used herein, the term “residue” when referring to α-amino acids is intended to mean a radical derived from the corresponding α-amino acid by eliminating the hydroxyl of the carboxy group and one hydrogen of the α-amino group. For example, the terms Gln, Ala, Gly, Ile, Arg, Asp, Phe, Ser, Leu, Cys, Asn, and Tyr represent the residues of L-glutamine, L-alanine, glycine, L-isoleucine, L-arginine, L-aspartic acid, L-phenylalanine, L-serine, L-leucine, L-cysteine, L-asparagine, and L-tyrosine, respectively.

As used herein, the term “subject” is intended to mean humans and non-human mammals such as primates, cats, dogs, swine, cattle, sheep, goats, horses, rabbits, rats, mice and the like.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

As used herein, the term “BIR domain binding” is intended to mean the action of a compound of the present invention upon an IAP BIR domain, which blocks or diminishes the binding of IAPs to BIR binding proteins or is involved in displacing BIR binding proteins from an IAP. Examples of BIR binding proteins include, but are not limited to, caspases and mitochondrially derived BIR binding proteins such as Smac, Omi/WTR2A and the like.

As used herein, the term “insufficient apoptosis” is intended to mean a state wherein a disease is caused or continues because cells deleterious to the subject have not apoptosed. This includes, but is not limited to, cancer cells that survive in a subject without treatment, cancer cells that survive in a subject during or following anti-cancer treatment, or immune cells whose action is deleterious to the subject, and includes, neutrophils, monocytes and auto-reactive T-cells.

As used herein, the term “therapeutically effective amount” is intended to mean an amount of a compound (e.g., a compound of Formula I, II, III, or IV described herein) which, when administered to a subject is sufficient to effect treatment for a disease-state associated with insufficient apoptosis. The amount of the compound will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state associated with insufficient apoptosis, as disclosed herein, in a subject, and includes: (i) preventing a disease or condition associated with insufficient apoptosis from occurring in a subject, in particular, when such mammal is predisposed to the disease or condition but has not yet been diagnosed as having it; (ii) inhibiting a disease or condition associated with insufficient apoptosis, i.e., arresting its development; or (iii) relieving a disease or condition associated with insufficient apoptosis, i.e., causing regression of the condition.

As used herein, the term “treating cancer” is intended to mean the administration of a pharmaceutical composition of the present invention to a subject, preferably a human, which is afflicted with cancer to cause an alleviation of the cancer by killing, inhibiting the growth, or inhibiting the metastasis of the cancer cells.

As used herein, the term “preventing disease” is intended to mean, in the case of cancer, the post-surgical, post-chemotherapy or post-radiotherapy administration of a pharmaceutical composition of the present invention to a subject, preferably a human, which was afflicted with cancer to prevent the regrowth of the cancer by killing, inhibiting the growth, or inhibiting the metastasis of any remaining cancer cells. Also included in this definition is the prevention of prosurvival conditions that lead to diseases such as asthma, MS and the like.

As used herein, the term “synergistic effect” is intended to mean that the effect achieved with the combination of the compounds of the present invention and either the chemotherapeutic agents or death receptor agonists of the invention is greater than the effect which is obtained with only one of the compounds, agents or agonists, or advantageously the effect which is obtained with the combination of the above compounds, agents or agonists is greater than the addition of the effects obtained with each of the compounds, agents or agonists used separately. Such synergy enables smaller doses to be given.

As used herein, the term “apoptosis” or “programmed cell death” is intended to mean the regulated process of cell death wherein a dying cell displays a set of well-characterized biochemical hallmarks that include cell membrane blebbing, cell soma shrinkage, chromatin condensation, and DNA laddering, as well as any caspase-mediated cell death.

As used herein, the term “BIR domain” or “BIR” are used interchangeably throughout and are intended to mean a domain which is characterized by a number of invariant amino acid residues including conserved cysteines and one conserved hisitidine residue within the sequence Cys-(Xaa1)₂Cys-(Xaa1)₁₆His-(Xaa1)₆₋₈Cys. Typically, the amino acid sequence of the consensus sequence is: Xaa1-Xaa1-Xaa1-Arg-Leu-Xaa1-Thr-Phe-Xaa1-Xaa1-Trp-Pro-Xaa2-Xaa1-Xaa1-Xaa2-Xaa2-Xaa1-Xaa1-Xaa1-Xaa1-Leu-Ala-Xaa1-Ala-Gly-Phe-Tyr-Tyr-Xaa1-Gly-Xaa1-Xaa1-Asp-Xaa1-Val-Xaa1-Cys-Phe-Xaa1-Cys-Xaa1-Xaa1-Xaa1-Xaa1-Xaa1-Xaa1-Trp-Xaa1-Xaa1-Xaa1-Asp-Xaa1-Xaa1-Xaa1-Xaa1-Xaa1-1-His-Xaa-1-Xaa1-Xaa1-Xaa1-Pro-Xaa1-Cys-Xaa1-Phe-Val, wherein Xaa1 is any amino acid and Xaa2 is any amino acid or is absent. Preferably the sequence is substantially identical to one of the BIR domain sequences provided for XIAP, HIAP1, or HIAP2 herein.

The BIR domain residues are listed below (see Genome Biology (2001) 1-10):

| | XIAP | HIAP-1 | HIAP-2 |
|---|---|---|---|
| BIR1 | 21-93 | 41-113 | 24-96 |
| BIR2 | 159-230 | 179-250 | 164-235 |
| BIR3 | 258-330 | 264-336 | 250-322 |
| Seq. # | P98170 | XP-006266 | XP-006267 |

As used herein, the term “ring zinc finger” or “RZF” is intended to mean a domain having the amino acid sequence of the consensus sequence: Glu-Xaa1-Xaa1-Xaa1-Xaa1-Xaa1-Xaa-1-Xaa2-Xaa1-Xaa1-Xaa1-Cys-Lys-Xaa3-Cys-Met-Xaa1-Xaa1-Xaa1-Xaa1-Xaa1-Xaa3-X-aa1-Phe-Xaa1-Pro-Cys-Gly-His-Xaa1-Xaa1-Xaa1-Cys-Xaa1-Xaa1-Cys-Ala-Xaa1-Xaa-1-Xaa1-Xaa1-Xaa1-Cys-Pro-Xaa1-Cys, wherein Xaa1 is any amino acid, Xaa2 is Glu or Asp, and Xaa3 is Val or Ile.

As used herein, the term “IAP” is intended to mean a polypeptide or protein, or fragment thereof, encoded by an IAP gene. Examples of IAPs include, but are not limited to human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apollon/BRUCE (Birc 6) (see for example U.S. Pat. Nos. 6,107,041; 6,133,437; 6,156,535; 6,541,457; 6,656,704; 6,689,562; Deveraux and Reed, Genes Dev. 13, 239-252, 1999; Kasof and Gomes, J. Biol. Chem., 276, 3238-3246, 2001; Vucic et al., Curr. Biol. 10, 1359-1366, 2000; Ashab et al. FEBS Lett., 495, 56-60, 2001, the contents of which are hereby incorporated by reference).

As used herein, the term “IAP gene” is intended to mean a gene encoding a polypeptide having at least one BIR domain and which is capable of modulating (inhibiting or enhancing) apoptosis in a cell or tissue. The IAP gene is a gene having about 50% or greater nucleotide sequence identity to at least one of human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apollon/BRUCE (Birc 6). The region of sequence over which identity is measured is a region encoding at least one BIR domain and a ring zinc finger domain. Mammalian IAP genes include nucleotide sequences isolated from any mammalian source.

As used herein, the term “IC₅₀” is intended to mean an amount, concentration or dosage of a particular compound of the present invention that achieves a 50% inhibition of a maximal response, such as displacement of maximal fluorescent probe binding in an assay that measures such response.

As used herein, the term “EC₅₀” is intended to mean an amount, concentration or dosage of a particular compound of the present invention that achieves a 50% inhibition of cell survival.

As used herein, the term “modulate” or “modulating” is intended to mean the treatment, prevention, suppression, enhancement or induction of a function or condition using the compounds of the present invention. For example, the compounds of the present invention can modulate IAP function in a subject, thereby enhancing apoptosis by significantly reducing, or essentially eliminating the interaction of activated apoptotic proteins, such as caspase-3, 7 and 9, with the BIR domains of mammalian IAPs or by inducing the loss of XIAP protein in a cell.

As used herein, the term “enhancing apoptosis” is intended to mean increasing the number of cells that apoptose in a given cell population either in vitro or in vivo. Examples of cell populations include, but are not limited to, ovarian cancer cells, colon cancer cells, breast cancer cells, lung cancer cells, pancreatic cancer cells, or T cells and the like. It will be appreciated that the degree of apoptosis enhancement provided by an apoptosis-enhancing compound of the present invention in a given assay will vary, but that one skilled in the art can determine the statistically significant change in the level of apoptosis that identifies a compound that enhances apoptosis otherwise limited by an IAP. Preferably “enhancing apoptosis” means that the increase in the number of cells undergoing apoptosis is at least 25%, more preferably the increase is 50%, and most preferably the increase is at least one-fold. Preferably the sample monitored is a sample of cells that normally undergo insufficient apoptosis (i.e., cancer cells). Methods for detecting the changes in the level of apoptosis (i.e., enhancement or reduction) are described in the Examples and include methods that quantitate the fragmentation of DNA, methods that quantitate the translocation phosphatoylserine from the cytoplasmic to the extracellular side of the membrane, determination of activation of the caspases and methods quantitate the release of cytochrome C and the apoptosis inhibitory factor into the cytoplasm by mitochondria.

As used herein, the term “proliferative disease” or “proliferative disorder” is intended to mean a disease that is caused by or results in inappropriately high levels of cell division, inappropriately low levels of apoptosis, or both. For example, cancers such as lymphoma, leukemia, melanoma, ovarian cancer, breast cancer, pancreatic cancer, and lung cancer, and autoimmune disoders are all examples of proliferative diseases.

As used herein, the term “death receptor agonist” is intended to mean an agent capable of stimulating by direct or indirect contact the pro apoptotic response mediated by the death-receptors. For example, an agonist TRAIL receptor antibody would bind to TRAIL receptor (S) and trigger an apoptotic response. On the other hand, other agent such as interferon-a could trigger the release of endogeneous TRAIL and/or up regulate the TRAIL receptors in such a way that the cell pro-apoptotic response is amplified.

The compounds of the present invention, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)- or (S)- or, as (D)- or (L)- for amino acids. The present invention is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, or selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively, specific enantiomers may be synthesized by asymmetric synthesis using optically active reagents, substrates, catalysts, or solvents or by converting one enantiomer to another by asymmetric transformation.

Certain compounds of the present invention may exist in Zwitterionic form and the present invention includes Zwitterionic forms of these compounds and mixtures thereof.

Utilities

The compounds of the present invention are useful as IAP BIR domain binding compounds and as such the compounds, compositions and method of the present invention include application to the cells or subjects afflicted with or having a predisposition towards developing a particular disease state, which is characterized by insufficient apoptosis. Thus, the compounds, compositions and methods of the present invention are used to treat cellular proliferative diseases/disorders, which include, but are not limited to, i) cancer, ii) autoimmune disease, iii) inflammatory disorders, iv) proliferation induced post medical procedures, including, but not limited to, surgery, angioplasty, and the like.

The compounds of the present invention may also be useful in the treatment of diseases in which there is a defect in the programmed cell-death or the apoptotic machinery (TRAIL, FAS, apoptosome), such as multiple sclerosis, artherosclerosis, inflammation, autoimmunity and the like.

The treatment involves administration to a subject in need thereof a compound of the present invention or a pharmaceutically acceptable salt thereof, or a pharmaceutical composition comprising a pharmaceutical carrier and a therapeutically effective amount of a compound of the present invention, or a pharmaceutically acceptable salt thereof. In particular, the compounds, compositions and methods of the present invention are useful for the treatment of cancer including solid tumors such as skin, breast, brain, lung, testicular carcinomas, and the like. Cancers that may be treated by the compounds, compositions and methods of the invention include, but are not limited to the following:

| Tissue | Example |
|---|---|
| Adrenal gland | neuroblastoma |
| Bone | osteogenic sarcoma (osteosarcoma), fibrosarcoma, malignant |
| | fibrous histiocytoma, chondrosarcoma, Ewing's sarcoma, |
| | malignant lymphoma (reticulum cell sarcoma), multiple |
| | myeloma, malignant giant cell tumor chordoma, |
| | osteochronfroma (osteocartilaginous exostoses), benign |
| | chondroma, chondroblastoma, chondromyxofibroma, osteoid |
| | osteoma and giant cell tumors |
| Cardiac | sarcoma (angiosarcoma, fibrosarcoma, rhabdomyosarcoma, |
| | liposarcoma), myxoma, rhabdomyoma, fibroma, lipoma and teratoma |
| Gastrointestinal | esophagus (squamous cell carcinoma, adenocarcinoma, |
| | leiomyosarcoma, lymphoma), stomach (carcinoma, lymphoma, |
| | leiomyosarcoma), pancreas (ductal adenocarcinoma, |
| | insulinoma, glucagonoma, gastrinoma, carcinoid tumors, |
| | vipoma), small bowel (adenocarcinoma, lymphoma, carcinoid |
| | tumors, Karposi's sarcoma, leiomyoma, hemangioma, lipoma, |
| | neurofibroma, fibroma), large bowel (adenocarcinoma, tubular |
| | adenoma, villous adenoma, hamartoma, leiomyoma) |
| Genitourinary | kidney (adenocarcinoma, Wilm's tumor [nephroblastoma], |
| tract | lymphoma, leukemia), bladder and urethra (squamous cell |
| | carcinoma, transitional cell carcinoma, adenocarcinoma), |
| | prostate (adenocarcinoma, sarcoma), testis (seminoma, |
| | teratoma, embryonal carcinoma, teratocarcinoma, |
| | choriocarcinoma, sarcoma, interstitial cell carcinoma, fibroma, |
| | fibroadenoma, adenomatoid tumors, lipoma) |
| Gynecological | uterus (endometrial carcinoma), cervix (cervical carcinoma, |
| | pre-tumor cervical dysplasia), ovaries (ovarian carcinoma |
| | [serous cystadenocarcinoma, mucinous cystadenocarcinoma, |
| | unclassified carcinoma], granulosa-thecal cell tumors, Sertoli- |
| | Leydig cell tumors, dysgerminoma, malignant teratoma), vulva |
| | (squamous cell carcinoma, intraepithelial carcinoma, |
| | adenocarcinoma, fibrosarcoma, melanoma), vagina (clear cell |
| | carcinoma, squamous cell carcinoma, botryoid sarcoma |
| | (embryonal rhabdomyosarcoma), fallopian tubes (carcinoma) |
| Hematologic | blood (myeloid leukemia [acute and chronic], acute |
| | lymphoblastic leukemia, chronic lymphocytic leukemia, |
| | myeloproliferative diseases, multiple myeloma, myelodysplastic |
| | syndrome), Hodgkin's disease, non-Hodgkin's lymphoma |
| | [malignant lymphoma] |
| Liver | hepatoma (hepatocellular carcinoma), cholangiocarcinoma, |
| | hepatoblastoma, angiosarcoma, hepatocellular adenoma, |
| | hemangioma |
| Lung | bronchogenic carcinoma (squamous cell, undifferentiated small |
| | cell, undifferentiated large cell, adenocarcinoma), alveolar |
| | (bronchiolar) carcinoma, bronchial adenoma, sarcoma, |
| | lymphoma, chondromatous hamartoma, mesothelioma |
| Nervous system | skull (osteoma, hemangioma, granuloma, xanthoma, osteitis |
| | deformans), meninges (meningioma, meningiosarcoma, |
| | gliomatosis), brain (astrocytoma, medulloblastoma, glioma, |
| | ependymoma, germinoma [pinealoma], glioblastoma multiform, |
| | oligodendroglioma, schwannoma, retinoblastoma, congenital |
| | tumors), spinal cord neurofibroma, meningioma, glioma, |
| | sarcoma) |
| Skin | malignant melanoma, basal cell carcinoma, squamous cell |
| | carcinoma, Karposi's sarcoma, moles dysplastic nevi, lipoma, |
| | angioma, dermatofibroma, keloids |

The compounds of the present invention, or their pharmaceutically acceptable salts or their prodrugs, may be administered in pure form or in an appropriate pharmaceutical composition, and can be carried out via any of the accepted modes of Galenic pharmaceutical practice.

The pharmaceutical compositions of the present invention can be prepared by mixing a compound of the present invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral (subcutaneous injections, intravenous, intramuscular, intrasternal injection or infusion techniques), sublingual, ocular, rectal, vaginal, and intranasal. Pharmaceutical compositions of the present invention are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound of the present invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the present invention, or a pharmaceutically acceptable salt thereof, for treatment of a disease-state as described above.

A pharmaceutical composition of the present invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

For oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the present invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; encapsulating agents such as cyclodextrins or functionalized cyclodextrins, including, but not limited to, α, β, or δ-hydroxypropylcyclodextins or Captisol; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

A liquid pharmaceutical composition of the present invention used for either parenteral or oral administration should contain an amount of a compound of the present invention such that a suitable dosage will be obtained. Typically, this amount is at least 0.01% of a compound of the present invention in the composition. When intended for oral administration, this amount may be varied to be between 0.1 and about 70% of the weight of the composition. For parenteral usage, compositions and preparations according to the present invention are prepared so that a parenteral dosage unit contains between 0.01 to 10% by weight of the compound of the present invention. Pharmaceutical compositions may be further diluted at the time of administration; for example a parenteral formulation may be further diluted with a sterile, isotonic solution for injection such as 0.9% saline, 5 wt % dextrose (D5W), Ringer's solution, or others.

The pharmaceutical composition of the present invention may be used for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the present invention from about 0.1 to about 10% w/v (weight per unit volume).

The pharmaceutical composition of the present invention may be used for rectal administration to treat for example, colon cancer, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the present invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the present invention in solid or liquid form may include an agent that binds to the compound of the present invention and thereby assists in the delivery of the compound. Suitable agents that may act in this capacity include, but are not limited to, a monoclonal or polyclonal antibody, a protein or a liposome.

The pharmaceutical composition of the present invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the present invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the present invention may be prepared by methodology well known in the pharmaceutical art. For example, a pharmaceutical composition intended to be administered by injection can be prepared by admixing a compound of the present invention with sterile, distilled water so as to form a solution. A surfactant may be added to facilitate the formation of a homogeneous solution or suspension. Surfactants are compounds that non-covalently interact with the compound of the present invention so as to facilitate dissolution or homogeneous suspension of the compound in the aqueous delivery system.

The compounds of the present invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of a compound of the present invention, or a pharmaceutically acceptable salt thereof.

Combination Therapy

The compounds of the present invention, or pharmaceutically acceptable salts thereof, may also be administered simultaneously with, prior to, or after administration of one or more of the therapeutic agents described below. Such combination therapy may include administration of a single pharmaceutical dosage formulation which contains a compound of the present invention and one or more additional agents given below, as well as administration of the compound of the present invention and of each additional agent in its own separate pharmaceutical dosage formulation. For example, a compound of the present invention and a chemotherapeutic agent, such as taxol (paclitaxel), taxotere, etoposide, cisplatin, vincristine, vinblastine, and the like, can be administered to the patient either together in a single oral dosage composition such as a tablet or capsule, or each agent administered in separate oral dosage formulations or via intravenous injection. Where separate dosage formulations are used, the compounds of the present invention and one or more additional agents can be administered at essentially the same time, i.e., concurrently, or at separately staggered times, i.e., sequentially; combination therapy is understood to include all these regimens. In addition, these compounds may synergize with molecules that may stimulate the death receptor apoptotic pathway through a direct or indirect manner. For example, the compounds of the present invention may be used in combination with soluble TRAIL, or in combination with procedures that can cause an increase in circulating level of TRAIL, such as interferon-alpha or radiation.

Thus, the present invention also encompasses the use of the compounds of the present invention in combination with radiation therapy or one or more additional agents such as those described in WO 03/099211 (PCT/US03/15861), which is hereby incorporated by reference.

Examples of such additional agents include, but are not limited to the following:
a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-.γ agonist,
m) a PPAR-.δ. agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug,
s) a proteasome inhibitor such as Velcade and MG132 (7-Leu-Leu-aldehyde) (see He at al. in Oncogene (2004) 23, 2554-2558),
t) an HDAC inhibitor, such as sodium butyrate, phenyl butyrate, hydroamic acids, cyclin tetrapeptide and the like (see Rosato et al,. Molecular Cancer Therapeutics 2003, 1273-1284),
u) an inhibitor of the chemotrypsin-like activity in the proteasome,
v) E3 ligase inhibitors,
w) a modulator of the immune system such as interferon-alpha and ionizing radition (UVB) that can induce the release of cytokines, such as the interleukins, TNF, or induce release of Death receptor Ligands such as TRAIL,
x) a modulator of TRAIL death receptors and TRAIL receptor agonists such as the humanized antibodies HGS-ETR1 and HGS-ETR2, and
or in combination or sequentially with radiation therapy, so as to treat the cancer.

Additional combinations may also include agents which reduce the toxicity of the aforesaid agents, such as hepatic toxicity, neuronal toxicity, nephprotoxicity and the like.

In one example, co-administration of one of the compounds of the present invention with a death receptor agonist such as TRAIL, such as a small molecule or an antibody that mimics TRAIL (e.g., a TRAIL receptor antibody) may cause an advantageous synergistic effect. Moreover, the compounds of the present invention may be used in combination with any compounds that cause an increase in circulating levels of TRAIL.

Vinca Alkaloids and Related Compounds

Vinca alkaloids that can be used in combination with the nucleobase oligomers of the invention to treat cancer and other neoplasms include vincristine, vinblastine, vindesine, vinflunine, vinorelbine, and anhydrovinblastine.

Dolastatins are oligopeptides that primarily interfere with tubulin at the vinca alkaloid binding domain. These compounds can also be used in combination with the compounds of the invention to treat cancer and other neoplasms. Dolastatins include dolastatin-10 (NCS 376128), dolastatin-15, ILX651, TZT-1027, symplostatin 1, symplostatin 3, and LU103793 (cemadotin).

Cryptophycins (e.g., cryptophycin 1 and cryptophycin 52 (LY355703)) bind tubulin within the vinca alkaloid-binding domain and induce G2/M arrest and apoptosis. Any of these compounds can be used in combination with the compounds of the invention to treat cancer and other neoplasms.

Other microtubule disrupting compounds that can be used in conjunction with the compounds of the invention to treat cancer and other neoplasms are described in U.S. Pat. Nos. 6,458,765; 6,433,187; 6,323,315; 6,258,841; 6,143,721; 6,127,377; 6,103,698; 6,023,626; 5,985,837; 5,965,537; 5,955,423; 5,952,298; 5,939,527; 5,886,025; 5,831,002; 5,741,892; 5,665,860; 5,654,399; 5,635,483; 5,599,902; 5,530,097; 5,521,284; 5,504,191; 4,879,278; and 4,816,444, and U.S. patent application Publication Nos. 2003/0153505 A1; 2003/0083263 A1; and 2003/0055002 A1, each of which is hereby incorporated by reference.

Taxanes and Other Micortubule Stabilizing Compounds

Taxanes such as paclitaxel, doxetaxel, RPR 109881A, SB-T-1213, SB-T-1250, SB-T-101187, BMS-275183, BRT 216, DJ-927, MAC-321, IDN5109, and IDN5390 can be used in combination with the compounds of the invention to treat cancer and other neoplasms. Taxane analogs (e.g., BMS-184476, BMS-188797) and functionally related non-taxanes (e.g., epothilones (e.g., epothilone A, epothilone B (EP0906), deoxyepothilone B, and epothilone B lactam (BMS-247550)), eleutherobin, discodermolide, 2-epi-discodermolide, 2-des-methyldiscodermolide, 5-hydroxymethyldiscoder-molide, 19-des-aminocarbonyldiscodermolide, 9(13)-cyclodiscodermolide, and laulimalide) can also be used in the methods and compositions of the invention.

Other microtubule stabilizing compounds that can be used in combination with the compounds of the invention to treat cancer and other neoplasms are described in U.S. Pat. Nos. 6,624,317; 6,610,736; 6,605,599; 6,589,968; 6,583,290; 6,576,658; 6,515,017; 6,531,497; 6,500,858; 6,498,257; 6,495,594; 6,489,314; 6,458,976; 6,441,186; 6,441,025; 6,414,015; 6,387,927; 6,380,395; 6,380,394; 6,362,217; 6,359,140; 6,306,893; 6,302,838; 6,300,355; 6,291,690; 6,291,684; 6,268,381; 6,262,107; 6,262,094; 6,147,234; 6,136,808; 6,127,406; 6,100,411; 6,096,909; 6,025,385; 6,011,056; 5,965,718; 5,955,489; 5,919,815; 5,912,263; 5,840,750; 5,821,263; 5,767,297; 5,728,725; 5,721,268; 5,719,177; 5,714,513; 5,587,489; 5,473,057; 5,407,674; 5,250,722; 5,010,099; and 4,939,168; and U.S. patent application Publication Nos. 2003/0186965 A1; 2003/0176710 A1; 2003/0176473 A1; 2003/0144523 A1; 2003/0134883 A1; 2003/0087888 A1; 2003/0060623 A1; 2003/0045711 A1; 2003/0023082 A1; 2002/0198256 A1; 2002/0193361 A1; 2002/0188014 A1; 2002/0165257 A1; 2002/0156110 A1; 2002/0128471 A1; 2002/0045609 A1; 2002/0022651 A1; 2002/0016356 A1; 2002/0002292 A1, each of which is hereby incorporated by reference.

Other chemotherapeutic agents that may be administered with a compound of the present invention are listed in the following Table:

| | | |
|---|---|---|
| Alkylating | cyclophosphamide | mechlorethamine |
| agents | lomustine | thiotepa |
| | busulfan | streptozocin |
| | procarbazine | chlorambucil |
| | ifosfamide | temozolomide |
| | altretamine | dacarbazine |
| | melphalan | semustine |
| | estramustine phosphate | carmustine |
| | hexamethylmelamine | |
| Platinum agents | cisplatin | tetraplatin |
| | carboplatinum | BBR-3464 (Hoffmann-La Roche) |
| | oxaliplatin | Ormiplatin |
| | ZD-0473 (AnorMED) | SM-11355 (Sumitomo) |
| | spiroplatinum | iproplatin |
| | lobaplatin (Aeterna) | AP-5280 (Access) |
| | carboxyphthalatoplatinum | |
| | satraplatin (Johnson Matthey) | |
| Antimetabolites | azacytidine | 6-mercaptopurine |
| | tomudex | hydroxyurea |
| | gemcitabine | 6-thioguanine |
| | trimetrexate | decitabine (SuperGen) |
| | capecitabine | cytarabin |
| | deoxycoformycin | clofarabine (Bioenvision) |
| | 5-fluorouracil | 2-fluorodeoxy |
| | fludarabine | cytidine |
| | floxuridine | irofulven (MGI Pharma) methotrexate |
| | pentostatin | DMDC (Hoffmann-La Roche) |
| | 2-chlorodeoxyadenosine | idatrexate |
| | raltitrexed | ethynylcytidine (Taiho) |
| Topoisomerase | amsacrine | TAS-103 (Taiho) |
| inhibitors | rubitecan (SuperGen) | Topotecan |
| | epirubicin | elsamitrucin (Spectrum) dexrazoxanet |
| | exatecan mesylate (Daiichi) | (TopoTarget) |
| | etoposide | J-107088 (Merck & Co) |
| | quinamed (ChemGenex) | pixantrone (Novuspharma) |
| | teniposide or mitoxantrone | BNP-1350 (BioNumerik) |
| | gimatecan (Sigma-Tau) | rebeccamycin analogue (Exelixis) |
| | irinotecan (CPT-11) | CKD-602 (Chong Kun Dang) |
| | diflomotecan (Beaufour-Ipsen) | BBR-3576 (Novuspharma) |
| | 7-ethyl-10-hydroxy-camptothecin | KW-2170 (Kyowa Hakko) |
| Antitumor | dactinomycin (actinomycin D) | bleomycinic acid |
| antibiotics | amonafide | idarubicin |
| | doxorubicin (adriamycin) | bleomycin A |
| | azonafide | rubidazone |
| | deoxyrubicin | bleomycin B |
| | anthrapyrazole | plicamycinp |
| | valrubicin | mitomycin C |
| | oxantrazole | porfiromycin |
| | daunorubicin (daunomycin) | MEN-10755 (Menarini) |
| | losoxantrone | cyanomorpholinodoxorubicin |
| | epirubicin | GPX-100 (Gem Pharmaceuticals) |
| | bleomycin sulfate (blenoxane) | mitoxantrone (novantrone) |
| | therarubicin | |
| Antimitotic | paclitaxel | RPR 109881A (Aventis) |
| agents | SB 408075 (GlaxoSmithKline) | ZD 6126 (AstraZeneca) |
| | docetaxel | TXD 258 (Aventis) |
| | E7010 (Abbott) | PEG-paclitaxel (Enzon) |
| | Colchicines | epothilone B (Novartis) |
| | PG-TXL (Cell Therapeutics) | AZ10992 (Asahi) |
| | vinblastine | T 900607 (Tularik) |
| | IDN 5109 (Bayer) | IDN-5109 (Indena) |
| | Vincristine | T 138067 (Tularik) |
| | A 105972 (Abbott) | AVLB (Prescient NeuroPharma) |
| | Vinorelbine | cryptophycin 52 (Eli Lilly) |
| | A 204197 (Abbott) | azaepothilone B (BMS) |
| | Vindesine | vinflunine (Fabre) |
| | LU 223651 (BASF) | BNP-7787 (BioNumerik) |
| | dolastatin 10 (NCI) | auristatin PE (Teikoku Hormone) |
| | D 24851 (ASTAMedica) | CA-4 prodrug (OXiGENE) |
| | rhizoxin (Fujisawa) | BMS 247550 (BMS) |
| | ER-86526 (Eisai) | dolastatin-10 (NIH) |
| | mivobulin (Warner-Lambert) | BMS 184476(BMS) |
| | combretastatin A4 (BMS) | CA-4 (OXiGENE) |
| | cemadotin (BASF) | BMS 188797 (BMS) |
| | isohomohalichondrin-B (PharmaMar) | taxoprexin (Protarga) |
| Aromatase | Aminoglutethimide | anastrazole |
| inhibitors | Exemestane | YM-511 (Yamanouchi) |
| | Letrozole | formestane |
| | atamestane (BioMedicines) | |
| Thymidylate | pemetrexed (Eli Lilly) | ZD-9331 (BTG) |
| synthase | nolatrexed (Eximias) | CoFactor ™ (BioKeys) |
| inhibitors | | |
| DNA | trabectedin (PharmaMar) | albumin + 32P (Isotope Solutions) |
| antagonists | mafosfamide (Baxter International) | O6 benzyl guanine (Paligent) |
| | glufosfamide (Baxter International) | thymectacin (NewBiotics) edotreotide |
| | apaziquone (Spectrum | (Novartis) |
| | Pharmaceuticals) | |
| Farnesyltransferase | arglabin (NuOncology Labs) | perillyl alcohol (DOR BioPharma) |
| inhibitors | tipifarnib (Johnson & Johnson) | BAY-43-9006 (Bayer) |
| | lonafarnib (Schering-Plough) | |
| Pump inhibitors | CBT-1 (CBA Pharma) | tariquidar (Xenova) |
| | zosuquidar trihydrochloride (Eli | biricodar dicitrate (Vertex) |
| | Lilly) | MS-209 (Schering AG) |
| Histone | tacedinaline (Pfizer) | depsipeptide (Fujisawa) |
| acetyltransferase | pivaloyloxymethyl butyrate (Titan) | MS-275 (Schering AG) |
| inhibitors | SAHA (Aton Pharma) | |
| Metalloproteinase | Neovastat (Aeterna Laboratories) | marimastat (British Biotech) BMS- |
| inhibitors | CMT-3 (CollaGenex) | 275291 (Celltech) |
| Ribonucleoside | gallium maltolate (Titan) | triapine (Vion) |
| reductase | tezacitabine (Aventis) | didox (Molecules for Health) |
| inhibitors | | |
| TNF alpha | virulizin (Lorus Therapeutics) | CDC-394 (Celgene) |
| agonists/antagonists | revimid (Celgene) | |
| Endothelin A | atrasentan (Abbott) | ZD-4054 (AstraZeneca) |
| receptor | YM-598 (Yamanouchi) | |
| antagonist | | |
| Retinoic acid | fenretinide (Johnson & Johnson) | LGD-1550 (Ligand) |
| receptor agonists | alitretinoin (Ligand) | |
| Immuno- | Interferon | norelin (Biostar) |
| modulators | dexosome therapy (Anosys) | IRX-2 (Immuno-Rx) |
| | oncophage (Antigenics) | BLP-25 (Biomira) |
| | pentrix (Australian Cancer | PEP-005 (Peplin Biotech) |
| | Technology) | MGV (Progenics) |
| | GMK (Progenics) | synchrovax vaccines (CTL Immuno) |
| | ISF-154 (Tragen) | beta.-alethine (Dovetail) |
| | adenocarcinoma vaccine (Biomira) | melanoma vaccine (CTL Immuno) |
| | cancer vaccine (Intercell) | CLL therapy (Vasogen) |
| | CTP-37 (A VI BioPharma) | p21 RAS vaccine (GemVax) |
| Hormonal and | estrogens | bicalutamide |
| antihormonal | Prednisone | testosterone propionate; |
| agents | conjugated estrogens | fluoxymesterone |
| | methylprednisolone | flutamide |
| | ethinyl estradiol | methyltestosterone |
| | prednisolone | octreotide |
| | chlortrianisen | diethylstilbestrol |
| | aminoglutethimide | nilutamide |
| | idenestrol | megestrol |
| | leuprolide | mitotane tamoxifen |
| | hydroxyprogesterone caproate | P-04 (Novogen) |
| | goserelin | Toremofine |
| | medroxyprogesterone | 2-methoxyestradiol (EntreMed) |
| | leuporelin | dexamethasone |
| | testosterone | arzoxifene (Eli Lilly) |
| Photodynamic | talaporfin (Light Sciences) | motexafin |
| agents | Pd-bacteriopheophorbide (Yeda) | gadolinium (Pharmacyclics) |
| | Theralux (Theratechnologies) | hypericin |
| | lutetium texaphyrin (Pharmacyclics) | |
| Tyrosine Kinase | imatinib (Novartis) | C225 (ImClone) |
| Inhibitors | kahalide F (PharmaMar) | ZD4190 (AstraZeneca) |
| | leflunomide (Sugen/Pharmacia) | rhu-Mab (Genentech) |
| | CEP-701 (Cephalon) | ZD6474 (AstraZeneca) |
| | ZD1839 (AstraZeneca) | MDX-H210 (Medarex) |
| | CEP-751 (Cephalon) | vatalanib (Novartis) |
| | erlotinib (Oncogene Science) | 2C4 (Genentech) |
| | MLN518 (Millenium) | PKI166 (Novartis) |
| | canertinib (Pfizer) | MDX-447 (Medarex) |
| | PKC412 (Novartis) | GW2016 (GlaxoSmithKline) |
| | squalamine (Genaera) | ABX-EGF (Abgenix) |
| | phenoxodiol ( ) | EKB-509 (Wyeth) |
| | SU5416 (Pharmacia) | IMC-1C11 (ImClone) |
| | trastuzumab (Genentech) | EKB-569 (Wyeth) |
| | SU6668 (Pharmacia) | |

Miscellaneous agents

| | |
|---|---|
| SR-27897 (CCK A inhibitor, Sanofi- | gemtuzumab (CD33 antibody, Wyeth Ayerst) |
| Synthelabo) | CCI-779 (mTOR kinase inhibitor, Wyeth) |
| BCX-1777 (PNP inhibitor, BioCryst) | PG2 (hematopoiesis enhancer, Pharmagenesis) |
| tocladesine (cyclic AMP agonist, Ribapharm) | exisulind (PDE V inhibitor, Cell Pathways) |
| ranpirnase (ribonuclease stimulant, Alfacell) | Immunol ™ (triclosan oral rinse, Endo) |
| alvocidib (CDK inhibitor, Aventis) | CP-461 (PDE V inhibitor, Cell Pathways) |
| galarubicin (RNA synthesis inhibitor, Dong-A) | triacetyluridine (uridine prodrug, Wellstat) |
| CV-247 (COX-2 inhibitor, Ivy Medical) | AG-2037 (GART inhibitor, Pfizer) |
| tirapazamine (reducing agent, SRI International) | SN-4071 (sarcoma agent, Signature BioScience) |
| P54 (COX-2 inhibitor, Phytopharm) | WX-UK1 (plasminogen activator inhibitor, |
| N-acetylcysteine (reducing agent, Zambon) | Wilex) |
| CapCell ™ (CYP450 stimulant, Bavarian | TransMID-107 .TM. (immunotoxin, KS |
| Nordic) | Biomedix) |
| R-flurbiprofen (NF-kappaB inhibitor, Encore) | PBI-1402 (PMN stimulant, ProMetic |
| GCS-100 (gal3 antagonist, GlycoGenesys) | LifeSciences) |
| 3CPA (NF-kappaB inhibitor, Active Biotech) | PCK-3145 (apoptosis promotor, Procyon) |
| G17DT immunogen (gastrin inhibitor, Aphton) | bortezomib (proteasome inhibitor, Millennium) |
| seocalcitol (vitamin D receptor agonist, Leo) | doranidazole (apoptosis promotor, Pola) |
| efaproxiral (oxygenator, Allos Therapeutics) | SRL-172 (T cell stimulant, SR Pharma) CHS- |
| 131-I-TM-601 (DNA antagonist, | 828 (cytotoxic agent, Leo) |
| TransMolecular) | TLK-286 (glutathione S transferase inhibitor, |
| PI-88 (heparanase inhibitor, Progen) | Telik) |
| eflornithine (ODC inhibitor, ILEX Oncology) | trans-retinoic acid (differentiator, NIH) |
| tesmilifene (histamine antagonist, YM | PT-100 (growth factor agonist, Point |
| BioSciences) | Therapeutics) |
| minodronic acid (osteoclast inhibitor, | MX6 (apoptosis promotor, MAXIA) |
| Yamanouchi) | midostaurin (PKC inhibitor, Novartis) |
| histamine (histamine H2 receptor agonist, | apomine (apoptosis promotor, ILEX Oncology) |
| Maxim) | bryostatin-1 (PKC stimulant, GPC Biotech) |
| indisulam (p53 stimulant, Eisai) | urocidin (apoptosis promotor, Bioniche) |
| tiazofurin (IMPDH inhibitor, Ribapharm) | CDA-II (apoptosis promotor, Everlife) |
| aplidine (PPT inhibitor, PharmaMar) | Ro-31-7453 (apoptosis promotor, La Roche) |
| cilengitide (integrin antagonist, Merck KGaA) | SDX-101 (apoptosis promotor, Salmedix) |
| rituximab (CD20 antibody, Genentech) | brostallicin (apoptosis promotor, Pharmacia) |
| SR-31747 (IL-1 antagonist, Sanofi-Synthelabo) | ceflatonin (apoptosis promotor, ChemGenex) |

Additional combinations may also include agents which reduce the toxicity of the aforesaid agents, such as hepatic toxicity, neuronal toxicity, nephprotoxicity and the like.

TRAIL Receptor Agonist Antibodies

Agonist antibodies directed against the death receptors TRAIL-R1 and/or TRAIL-R2 can be used in combination with compounds of the invention. Exemplary agonist antibodies that may be used in combination with compounds of the invention include those described in U.S. Pat. No. 7,244,429; in U.S. Patent Application Publication Nos. 2007/0179086, 2002/0004227, 2006/0269554 , 2005/0079172, 2007/0292411, 2006/0270837, 2006/0269555, 2004/0214235, and 2007/0298039; and in International Patent Publications WO2006/017961 and WO98/51793. Each of these publications is hereby incorporated by reference in its entirety. In preferred embodiments, compounds of the invention are used in combination with one or more of these TRAIL receptor agonist antibodies for the treatment of cancer and other neoplasms.

Screening Assays

The compounds of the present invention may also be used in a method to screen for other compounds that bind to an IAP BIR domain. Generally speaking, to use the compounds of the invention in a method of identifying compounds that bind to an IAP BIR domain, the IAP is bound to a support, and a compound of the invention is added to the assay. Alternatively, the compound of the invention may be bound to the support and the IAP is added.

There are a number of ways in which to determine the binding of a compound of the present invention to the BIR domain. In one way, the compound of the invention, for example, may be fluorescently or radioactively labeled and binding determined directly. For example, this may be done by attaching the IAP to a solid support, adding a detectably labeled compound of the invention, washing off excess reagent, and determining whether the amount of the detectable label is that present on the solid support. Numerous blocking and washing steps may be used, which are known to those skilled in the art.

In some cases, only one of the components is labeled. For example, specific residues in the BIR domain may be labeled. Alternatively, more than one component may be labeled with different labels; for example, using I¹²⁵ for the BIR domain, and a fluorescent label for the probe.

The compounds of the invention may also be used as competitors to screen for additional drug candidates or test compounds. As used herein, the terms “drug candidate” or “test compounds” are used interchangeably and describe any molecule, for example, protein, oligopeptide, small organic molecule, polysaccharide, polynucleotide, and the like, to be tested for bioactivity. The compounds may be capable of directly or indirectly altering the IAP biological activity.

Drug candidates can include various chemical classes, although typically they are small organic molecules having a molecular weight of more than 100 and less than about 2,500 Daltons. Candidate agents typically include functional groups necessary for structural interaction with proteins, for example, hydrogen bonding and lipophilic binding, and typically include at least an amine, carbonyl, hydroxyl, ether, or carboxyl group. The drug candidates often include cyclical carbon or heterocyclic structures and/or aromatic or polyaromatic structures substituted with one or more functional groups.

Drug candidates can be obtained from any number of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Competitive screening assays may be done by combining an IAP BIR domain and a probe to form a probe:BIR domain complex in a first sample followed by adding a test compound from a second sample. The binding of the test is determined, and a change or difference in binding between the two samples indicates the presence of a test compound capable of binding to the BIR domain and potentially modulating the IAP's activity.

In one case, the binding of the test compound is determined through the use of competitive binding assays. In this embodiment, the probe is labeled with a fluorescent label. Under certain circumstances, there may be competitive binding between the test compound and the probe. Test compounds which display the probe, resulting in a change in fluorescence as compared to control, are considered to bind to the BIR region.

In one case, the test compound may be labeled. Either the test compound, or a compound of the present invention, or both, is added first to the IAP BIR domain for a time sufficient to allow binding to form a complex.

Formation of the probe:BIR domain complex typically requires incubations of between 4° C. and 40° C. for between 10 minutes to about 1 hour to allow for high-throughput screening. Any excess of reagents are generally removed or washed away. The test compound is then added, and the presence or absence of the labeled component is followed, to indicate binding to the BIR domain.

In one case, the probe is added first, followed by the test compound. Displacement of the probe is an indication the test compound is binding to the BIR domain and thus is capable of binding to, and potentially modulating, the activity of IAP. Either component can be labeled. For example, the presence of probe in the wash solution indicates displacement by the test compound. Alternatively, if the test compound is labeled, the presence of the probe on the support indicates displacement.

In one case, the test compound may be added first, with incubation and washing, followed by the probe. The absence of binding by the probe may indicate the test compound is bound to the BIR domain with a higher affinity. Thus, if the probe is detected on the support, coupled with a lack of test compound binding, this may indicate the test compound is capable of binding to the BIR domain.

Modulation is tested by screening for a test compound's ability to modulate the activity of IAP and includes combining a test compound with an IAP BIR domain, as described above, and determining an alteration in the biological activity of the IAP. Therefore in this case, the test compound should both bind to the BIR domain (although this may not be necessary), and alter its biological activity as defined herein.

Positive controls and negative controls may be used in the assays. All control and test samples are performed multiple times to obtain statistically significant results. Following incubation, all samples are washed free of non-specifically bound material and the amount of bound probe determined. For example, where a radiolabel is employed, the samples may be counted in a scintillation counter to determine the amount of bound compound.

Typically, the signals that are detected in the assay may include fluorescence, resonance energy transfer, time resolved fluorescence, radioactivity, fluorescence polarization, plasma resonance, or chemiluminescence and the like, depending on the nature of the label. Detectable labels useful in performing screening assays in this invention include a fluorescent label such as Fluorescein, Oregon green, dansyl, rhodamine, tetramethyl rhodamine, texas red, Eu³⁺; a chemiluminescent label such as luciferase; colorimetric labels; enzymatic markers; or radioisotopes such as tritium, I¹²⁵ and the like.

Affinity tags, which may be useful in performing the screening assays of the present invention, include biotin, polyhistidine and the like.

### Synthesis and Methodology

Scheme 1 summarizes the synthesis of compound 13.

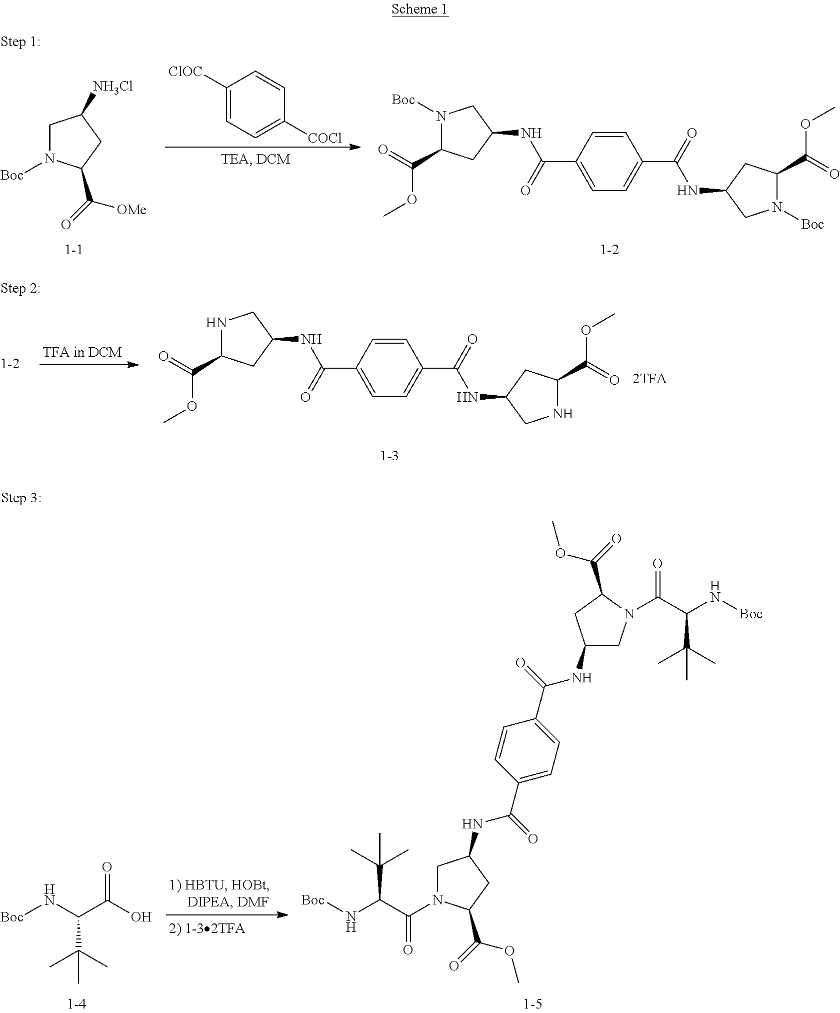

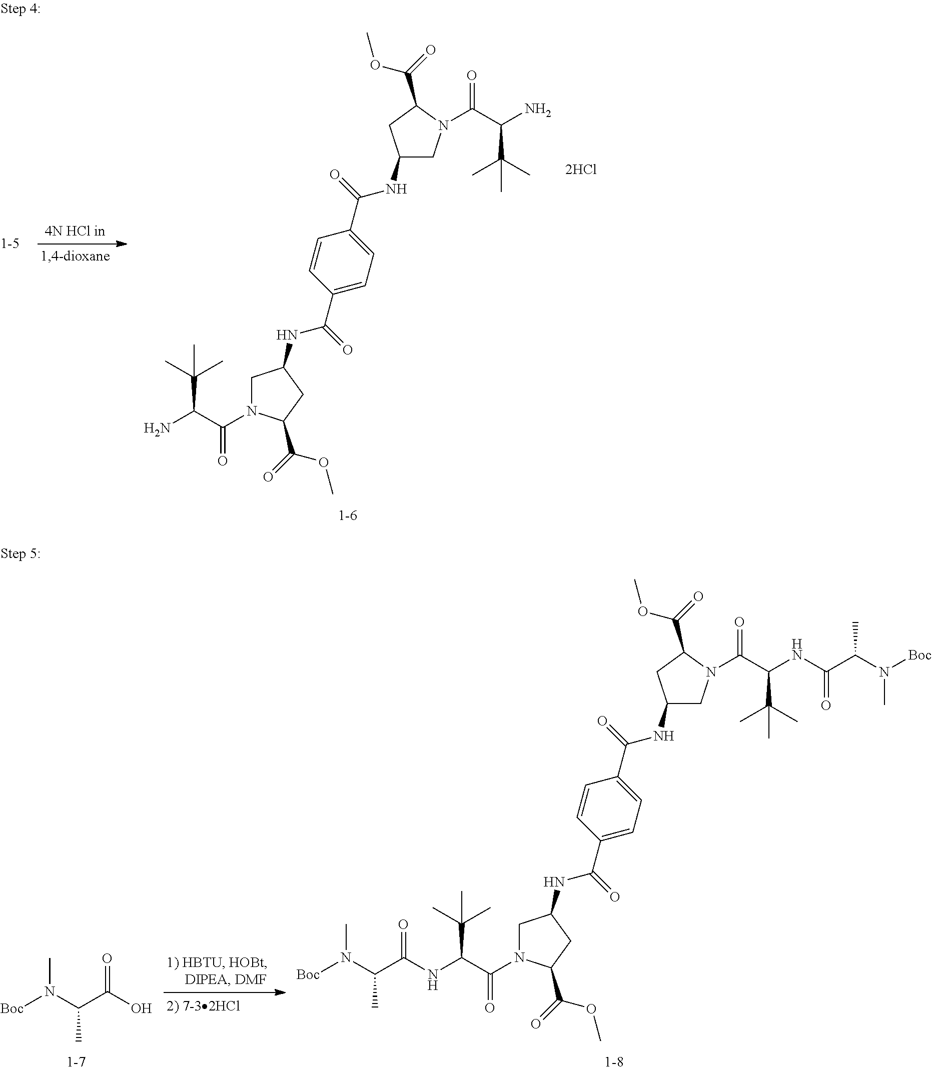

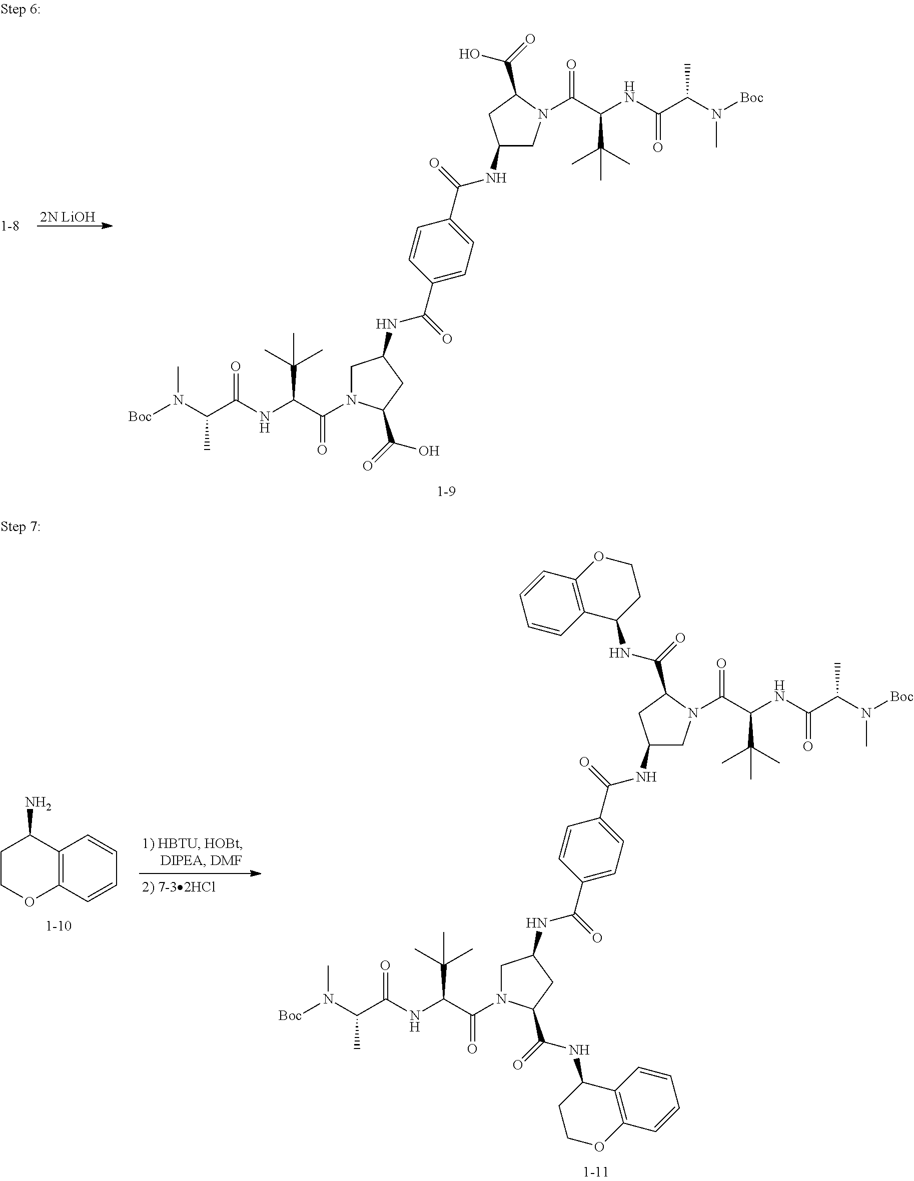

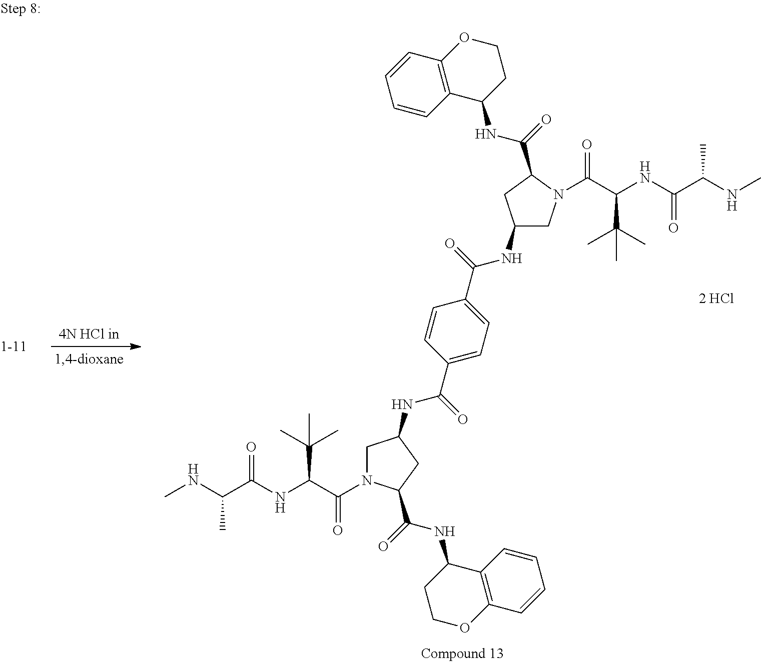

Preparative Methods

Step 1: Intermediate 1-2

To a solution of N-Boc-cis-4-amino-L-proline methyl ester hydrochloride, 1-1, (25.0 g, 89.2 mmol), in CH₂Cl₂ cooled to 0° C., were sequentially added triethylamine (50.0 mL, 356.8 mmol), DMAP (545 mg, 4.46 mmol) and terephthaloyl chloride (8.69 g, 42.8 mmol). The reaction was stirred overnight at room temperature. Water and ethyl acetate were added, the organic layer was separated, washed with 10% citric acid, aqueous NaHCO₃ and brine, dried over anhydrous MgSO₄, filtered and concentrated in vacuo. Purification by silica gel chromatography provided intermediate 1-2 as a pale yellow solid.

Step 2: Intermediate 1-3

Intermediate 1-2 (8.03 g) was dissolved in a mixture of CH₂Cl₂ (65 mL) and TFA (65 mL) at 0° C. The solution was stirred for 1 hour at 0° C. and for 5 hours at room temperature. Volatiles were removed under reduced pressure and the residue was triturated with diethyl ether to provide intermediate 1-3•2TFA as a white solid. MS (m/z) M+1=419.2

Step 3: Intermediate 1-5

To a solution of Boc-Tle-OH 1-4 (6.60 g, 28.6 mmol) in DMF cooled to 0° C. were sequentially added, DIPEA (18.13 ml, 104 mmol), HOBt (4.03 g, 29.8 mmol) and HBTU (11.32 g, 29.8 mmol). After stirring for 10 minutes intermediate 1-3•2TFA (8.39 g, 12.9 mmol) was added and the reaction mixture was stirred overnight at room temperature. Water and ethyl acetate were added, the organic layer was separated, washed with 10% citric acid, saturated NaHCO₃, and brine, dried over anhydrous MgSO₄, filtered and concentrated in vacuo. Purification by silica gel chromatography provided intermediate 1-5 as a white solid.

Step 4: Intermediate 1-6

4N HCl in 1,4-dioxane (20 ml) was added to intermediate 1-5 (9.9 g, 11.7 mmol) and the suspesion was stirred at 0° C. for 2 hours. Volatiles were removed under reduced pressure and the residue was triturated with diethyl ether to provide intermediate 1-6•2HCl as a white solid. MS (m/z) M+1=645.5

Step 5: Intermediate 1-8

To a solution of Boc-NMe-Ala-OH 1-7, (5.89 g, 29.0 mmol) in DMF cooled to 0° C. were sequentially added, DIPEA (19.47 ml, 111.0 mmol), HOBt (4.52 g, 33.4 mmol) and HBTU (12.68 g, 33.4 mmol). After stirring for 10 minutes intermediate 1-6•2HCl (8.0 g, 11.1 mmol) was added and the reaction mixture was stirred overnight at room temperature. Water and ethyl acetate were added; the organic layer was separated, washed with 10% citric acid, saturated NaHCO₃, and brine, dried over anhydrous MgSO4, filtered and concentrated in vacuo. Purification by silica gel chromatography provided intermediate 1-8 as a white solid.

Step 6: Intermediate 1-9

To a solution of intermediate 1-8 (5.08 g, 5.0 mmol) in THF (25 mL) cooled to 0° C. was added 2N aqueous LiOH (25 mL, 50.0 mmol) and the reaction was stirred overnight at room temperature. The pH was adjusted to 3 with 10% citric acid and ethyl acetate was added. The organic layer was separated and the aqueous phase was extracted two times with ethyl acetate. The combined organic layers were washed with brine, dried over anhydrous MgSO₄, filtered and concentrated in vacuo to provide intermediate 1-9 as a white solid.

Step 7: Intermediate 1-11

To a solution of intermediate 1-9 (493 mg, 0.49 mmol) in DMF cooled to 0° C. were sequentially added, DIPEA (0.87 ml, 5.0 mmol), HOBt (202 mg, 1.49 mmol) and HBTU (568 mg, 1.49 mmol). After stirring for 10 minutes (R)-chroman-4-amine-HCl salt 1-10 (241 mg, 1.29 mmol) was added and the reaction mixture was stirred overnight at room temperature. Water and ethyl acetate were added, the organic layer was separated, washed with 10% citric acid, saturated NaHCO₃, and brine, dried over anhydrous MgSO₄, filtered and concentrated in vacuo. Purification by silica gel chromatography provided intermediate 1-11 as a beige solid.

Step 7: Compound 13

4N HCl in 1,4-dioxane (1.0 ml) was added to intermediate 1-11 (250 mg, 0.2 mmol) and the suspesion was stirred at 0° C. for 2 hours. Volatiles were removed under reduced pressure and the residue was triturated with diethyl ether to provide compound 13•2HCl as a white solid. MS (m/z) M+1=1049.4

Mass Spectrum (MS) characterization of disclosed compounds is listed below.

| Compound | MS (m/z) |
|---|---|
| 1 | [M + 2H]²⁺ = 625.6 |
| 2 | [M + 2H]²⁺ = 653.6 |
| 3 | [M + 2H]²⁺ = 631.6 |
| 4 | [M + 2H]²⁺ = 583.6 |
| 5 | [M + 2H]²⁺ = 659.6 |
| 6 | [M + 2H]²⁺ = 617.6 |
| 7 | [M + 2H]²⁺ = 603.6 |
| 8 | [M + 2H]²⁺ = 541.4 |
| 9 | [M + 2H]²⁺ = 573.8 |
| 10 | [M + 2H]²⁺ = 589.6 |
| 11 | [M + 2H]²⁺ = 509.2 |
| 12 | [M + 2H]²⁺ = 537.3 |
| 14 | [M + 2H]²⁺ = 541.2 |
| 15 | [M + 2H]²⁺ = 573.2 |

Cell Culture and Cell Death Assays

A. Cell Culture

SKOV3 (ovarian) and HCT-116 (colon) cancer cells were cultured in McCoy's 5A media supplemented with 10% FBS and 100 units/mL of Penicillin and Streptomycin.

B. Assays

Cytotoxicity assays were performed on SKOV3 and HCT-116 cell lines. Cells were seeded in 96 well plates at a respective density of 2000 and 5000 cells per well and incubated at 37° C. in presence of 5% CO₂ for 24 hours. Selected compounds were diluted into the media at various concentrations ranging from 0.01 nM up to 100 nM. Diluted compounds were added onto the SKOV3 cells. HCT-116 cells were co-treated with Trail receptor monoclonal antibody agonist (ETR1, 40 ng/mL). After 72 hours, cellular viability was evaluated by MTT conversion. A solution of 3-(4,5-dimethylthiazol-2-yl)-2,5-diphenyltetrazolium bromide (MU) was added onto cells for a period of 1 to 4 hours. Media was removed and replaced with isopropanol. Conversion of MTT by viable cells was detected by absorbance at 570 nM. The percentage of viability was expressed in percentage of the signal obtained with non treated cells.

Select compounds of the present invention are cytotoxic to SKOV3 cells with EC₅₀ values of 100 nM or less. EC₅₀ values for cytotoxicity of compounds of the present invention to HCT116 cells in the presence of agonist Trail receptor monoclonal antibody ETR1 is in the range of 50 nM or less.

Compounds represented hereinabove potently killed SKOV-3 ovarian cancer cells. Further compounds of the instant invention reduced the viability of HCT116 colon cancer cells in the presence of ETR1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of by Formula I: 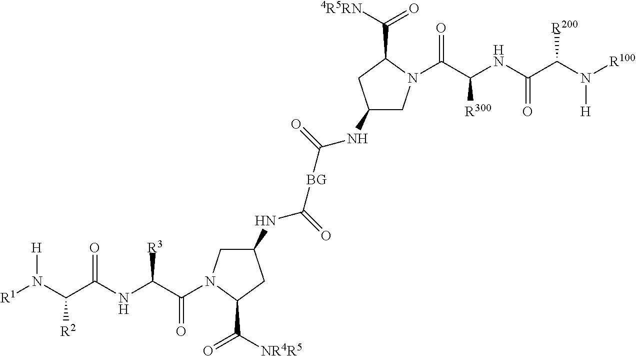
or a salt thereof, wherein:
BG is cyclohexyl, phenyl, naphthyl or biphenyl optionally substituted with R¹⁰;
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents
R⁴ is H or C₁-C₆ alkyl;
R⁵ is 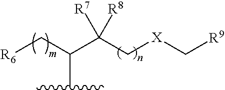
wherein:
m is 0 or 1;
n is 0 or 1;
X is O, S, or SO₂;
R⁶ is C₆-C₁₀ aryl optionally substituted with one or more R¹¹ substituents;
R⁷ and R⁸ are both hydrogen or taken together form (═O); and
R⁹ is
1) C₁-C₁₀ alkyl optionally substituted with one or more R^{I}° substituents;
2) naphthyl optionally substituted with one or more R¹⁰ substituents; or
3) phenyl substituted with one or more R¹⁰ substituents;
or R⁵ is 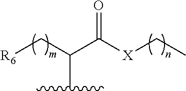
wherein:
m is 0 or 1;
n is 0-6;
X is O or S; and
R⁶ is C₆-C₁₀ aryl optionally substituted with one or more R¹¹ substituents;
R¹⁰ is C₁-C₆ alkyl or C₆-C₁₀ aryl optionally substituted with C₁-C₆ alkyl;
R¹¹ is
1) halogen,
2) OR¹²,
3) SR¹²,
4) NO₂,
5) CN,
6) haloalkyl,
7) C₁-C₆ alkyl,
8) C₂-C₆ alkenyl,
9) C₂-C₄ alkynyl,
10) C₃-C₇ cycloalkyl,
11) C₃-C₇ cycloalkenyl, or
12) C₆-C₁₀ aryl;
and R¹² is C₁-C₆ alkyl.

2. The compound of claim 1, wherein R⁵ is 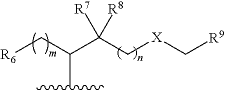
and R⁷ and R⁸ are both hydrogen.

3. The compound of claim 2, wherein the compound is

| Cmpd. | Structure |
|---|---|
| 1 | 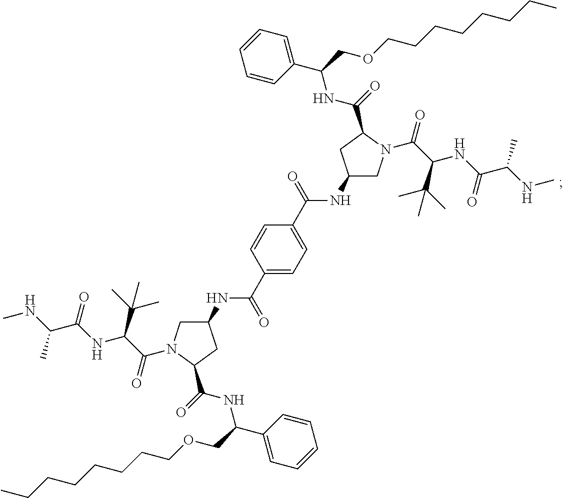 |
| 2 | 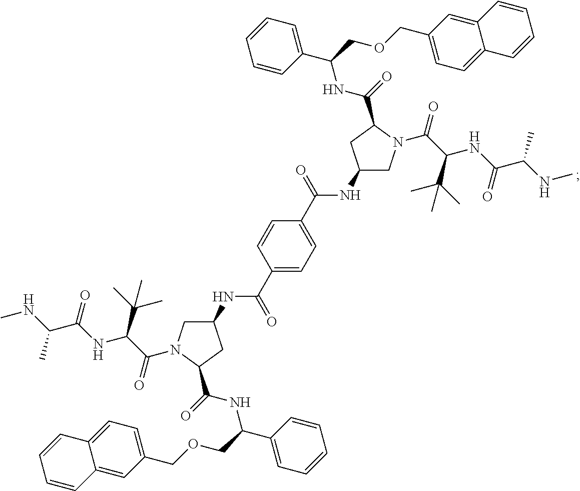 |
| 3 | 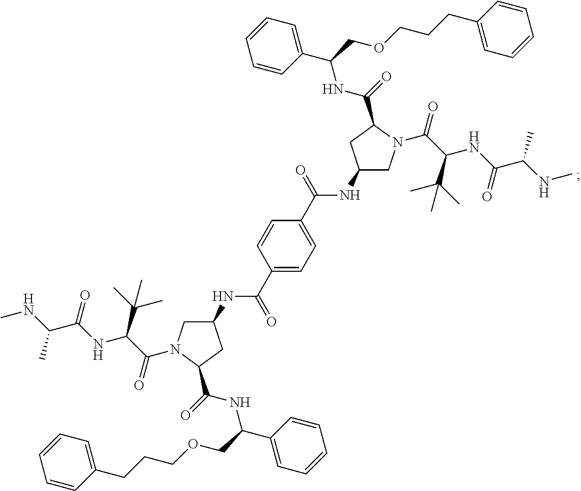 |
| 4 | 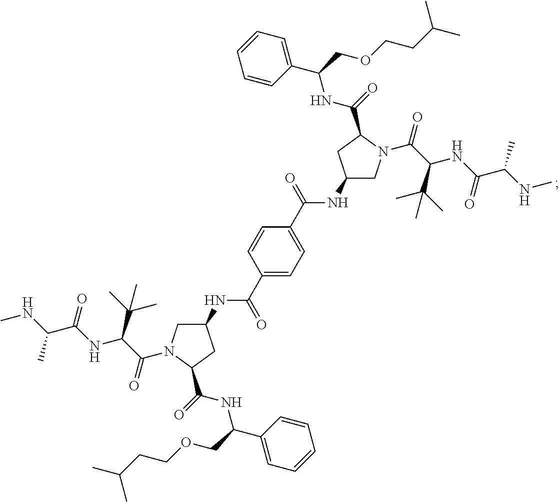 |
| 5 | 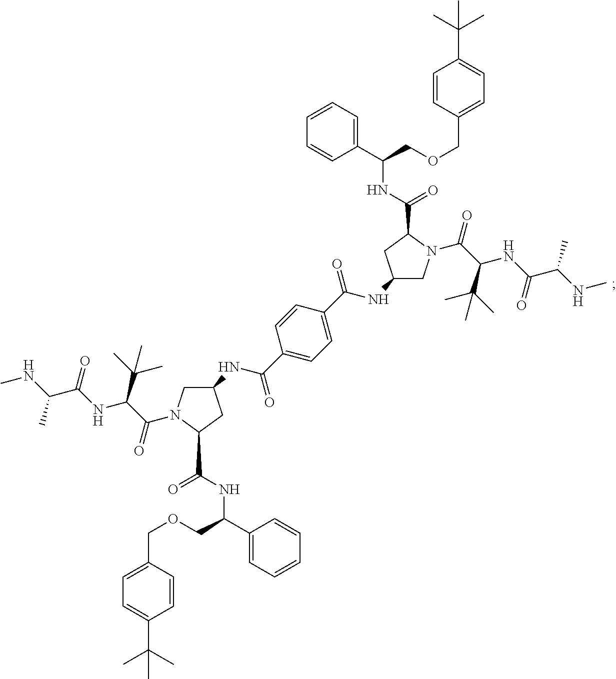 |
| 6 | 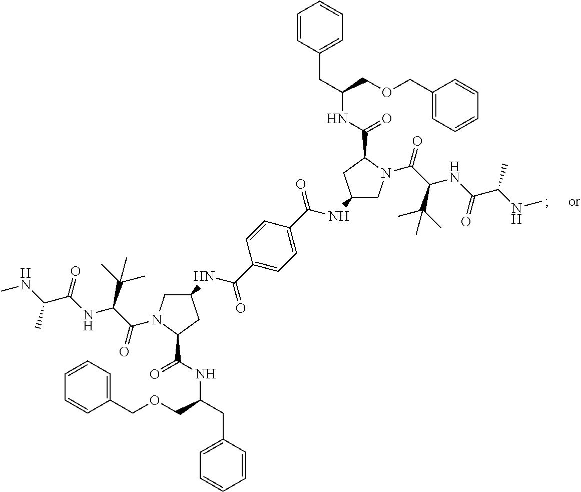 |
| 7 | 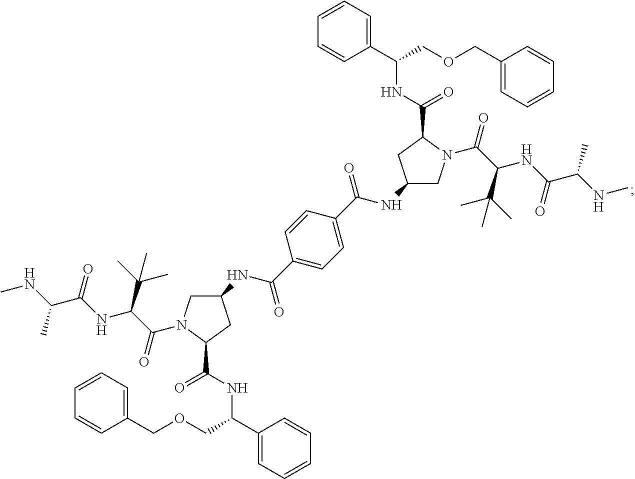 |

or a salt thereof.

4. (canceled)

5. The compound of claim 1, wherein the compound is 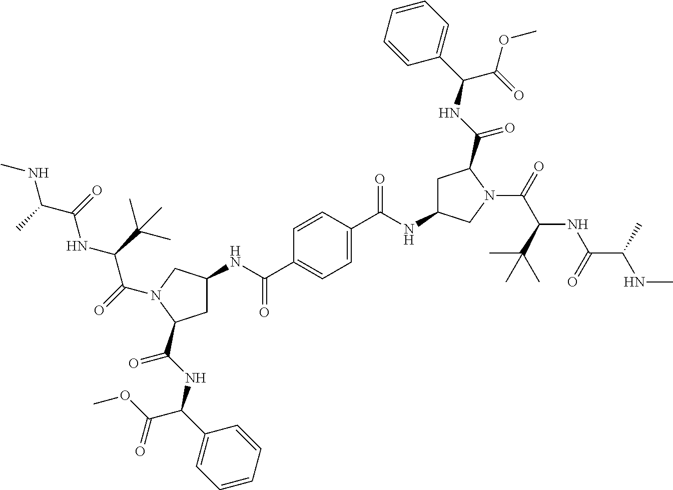
or salt thereof.

6. A compound of Formula II: 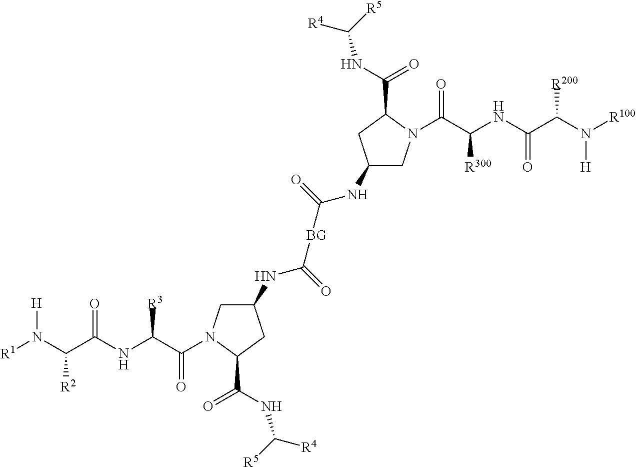
or a salt thereof, wherein:
BG is cyclohexyl, phenyl, naphthyl or biphenyl optionally substituted with R¹⁰;
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents;
R⁴ is C₆-C₁₀ aryl substituted with R¹¹;
R⁵ is C₆-C₁₀ aryl optionally substituted with R¹¹, wherein R⁴ and R⁵ are not the same;
R¹⁰ is C₁-C₆ alkyl or C₆-C₁₀ aryl optionally substituted with C₁-C₆ alkyl;
R¹¹ is
1) halogen,
2) OR¹²,
3) SR¹²,
4) NO₂,
5) CN,
6) haloalkyl,
7) C₁-C₆ alkyl,
8) C₂-C₆ alkenyl,
9) C₂-C₄ alkynyl,
10) C₃-C₇ cycloalkyl,
11) C₃-C₇ cycloalkenyl, or
12) C₆-C₁₀ aryl;
and R¹² is C₁-C₆ alkyl.

7. The compound of claim 6, wherein the compound is

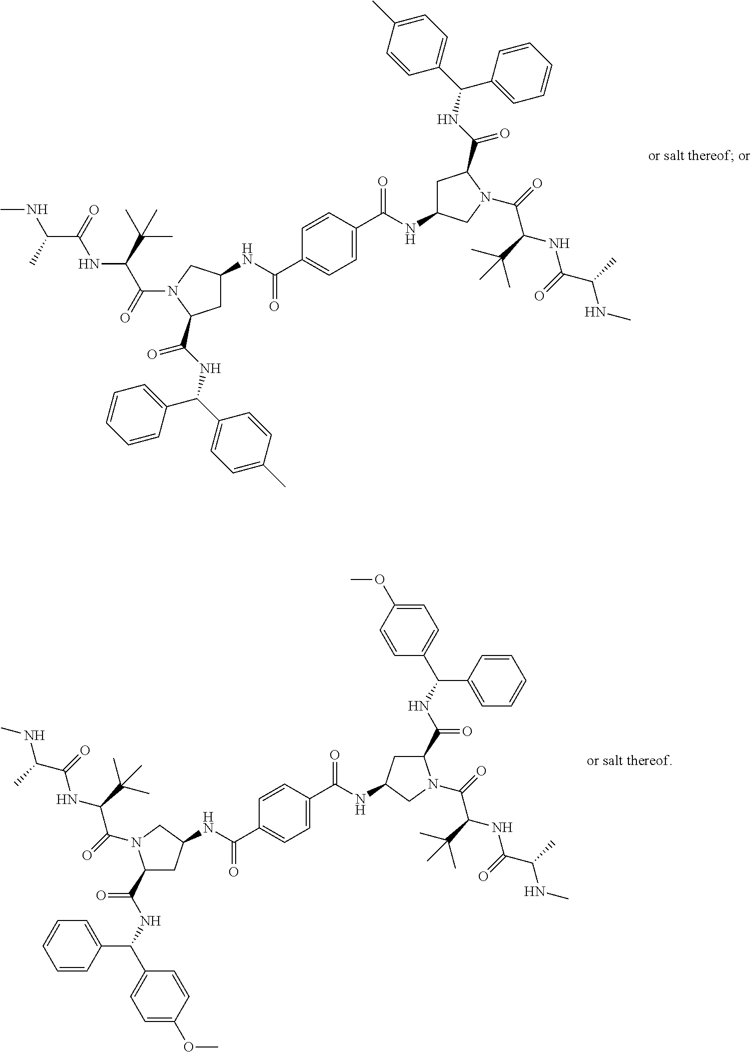

8. A compound of by Formula III: 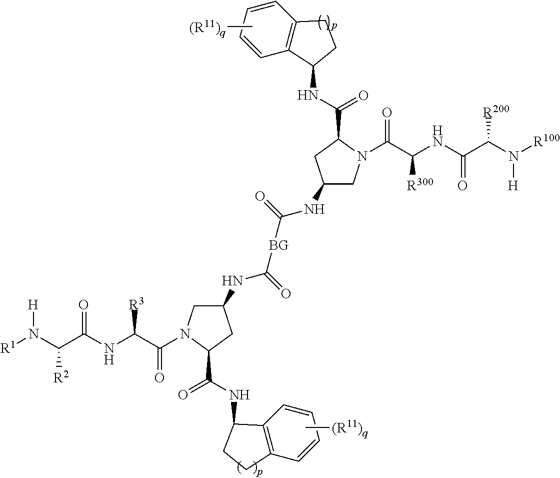
or a salt thereof,
or Formula IV: 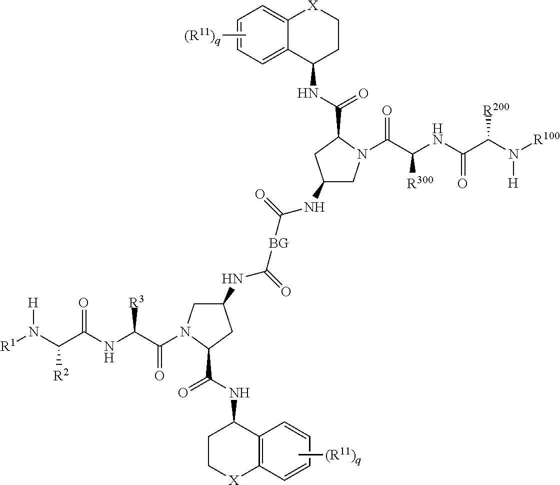
or a salt thereof,
or Formula V: 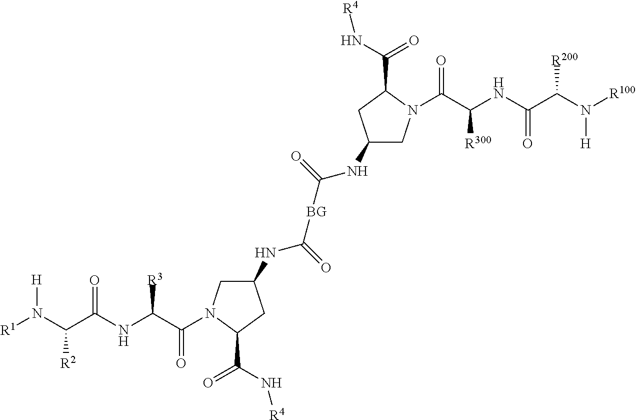
or a salt thereof,
wherein:
BG is cyclohexyl, phenyl, naphthyl or biphenyl optionally substituted with R¹⁰;
R¹ and R¹⁰⁰ are independently H or C₁-C₆ alkyl optionally substituted with one or more R¹¹ substituents;
R² and R²⁰⁰ are independently C₁-C₃ alkyl optionally substituted with halogen;
R³ and R³⁰⁰ are independently C₁-C₆ alkyl optionally substituted with one or more R¹¹ substituents;
R⁴ is aryl or heteroaryl which is further substituted with a heteroaryl;
p is 1 or 3;
q is 0 to 4;
X is O, S, or SO₂;
R¹⁰ is C₁-C₆ alkyl or C₆-C₁₀ aryl optionally substituted with C₁-C₆ alkyl;
R¹¹ is
1) halogen,
2) OR¹²,
3) SR¹²,
4) NO₂,
5) CN,
6) haloalkyl,
7) C₁-C₆ alkyl,
8) C₂-C₆ alkenyl,
9) C₂-C₄ alkynyl,
10) C₃-C₇ cycloalkyl,
11) C₃-C₇ cycloalkenyl, or
12) C₆-C₁₀ aryl;
and R¹² is C₁-C₆ alkyl.

9. The compound of claim 8, wherein the compound is

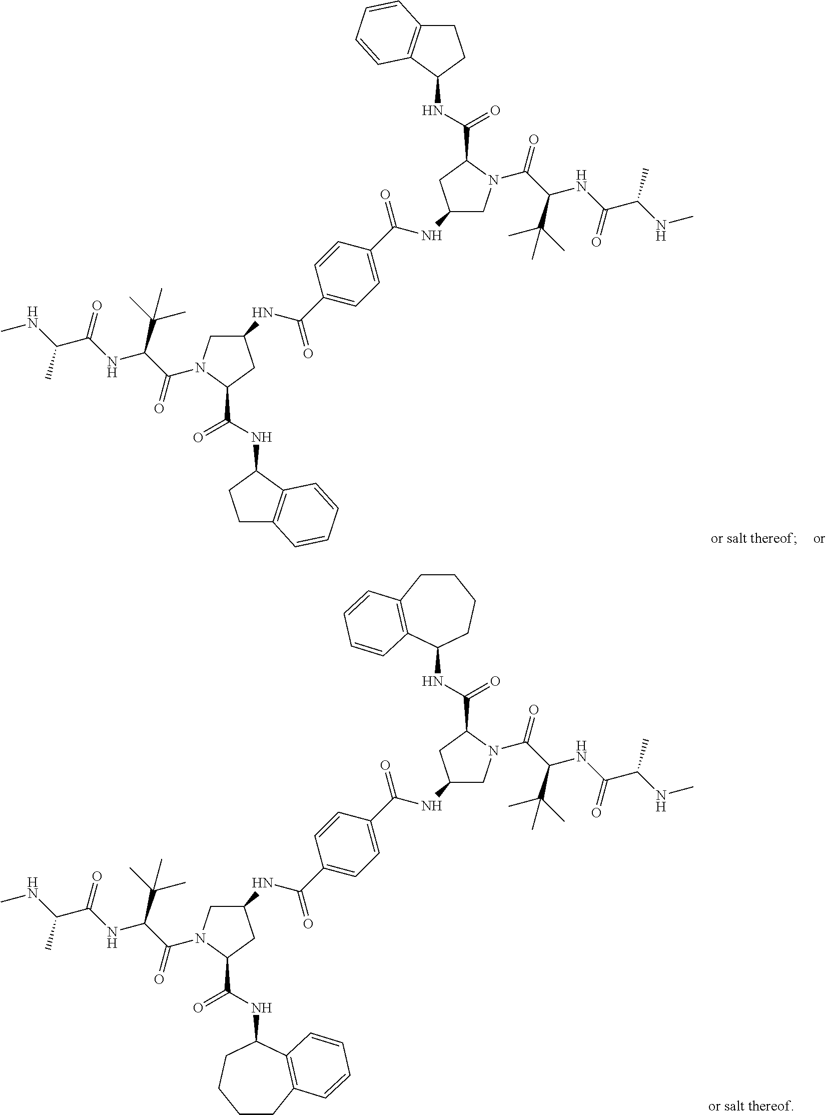

10. (canceled)

11. The compound of claim 8, wherein the compound is

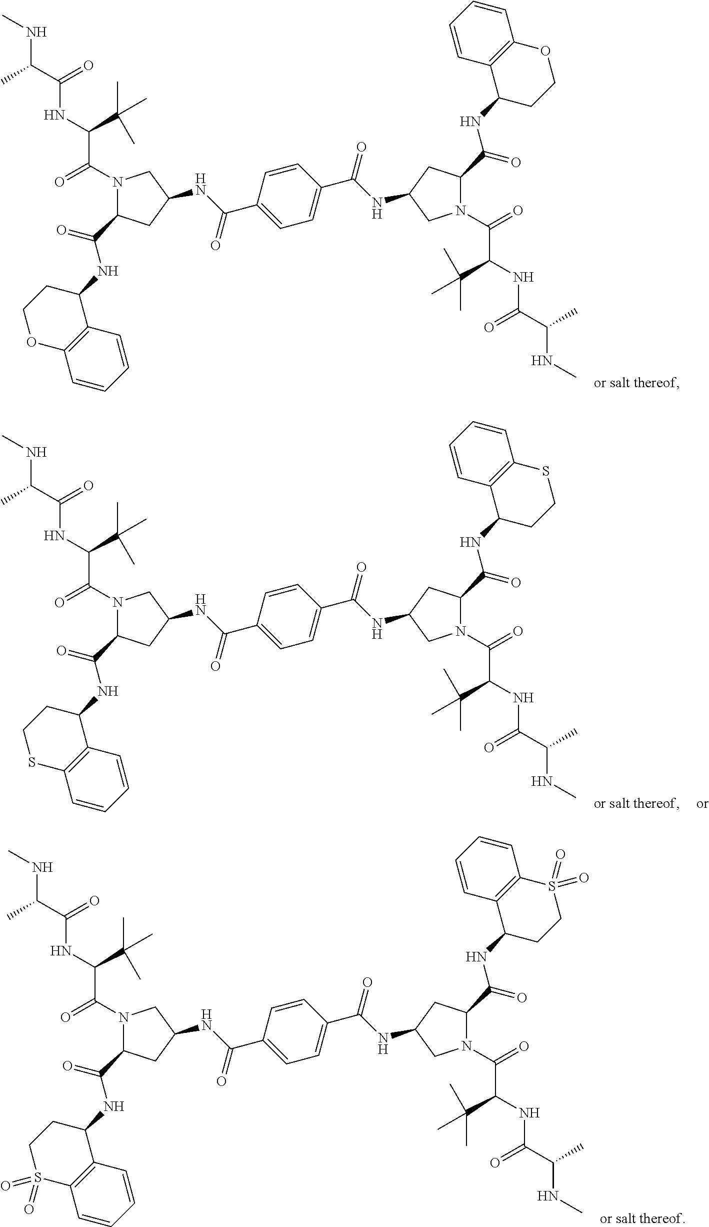

12. (canceled)

13. The compound of claim 8, wherein the compound is of Formula V and R⁴ is

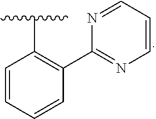

14. The compound of claim 8, wherein the compound is 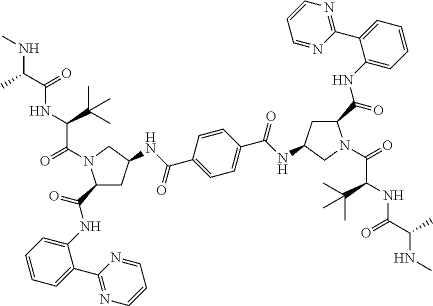
or salt thereof.

15. The compound of claim 1, wherein BG is phenyl.

16. (canceled)

17. A pharmaceutical composition comprising the compound of claim 1 and a pharmaceutically acceptable carrier, diluent, or excipient.

18. The pharmaceutical composition of claim 17 further comprising a chemotherapeutic agent or a death receptor agonist.

19. The pharmaceutical composition of claim 18, wherein the death receptor agonist is TRAIL or a TRAIL receptor antibody.

20. A method for the treatment or prevention of a proliferative disorder in a subject, the method comprising administering to the subject a compound of claim 1.

21. The method of claim 20, wherein the proliferative disorder is cancer.

22. The method of claim 20, further comprising administering to the subject a chemotherapeutic agent prior to, simultaneously with, or after administration of the compound of claim 1.

23. The method of claim 20, further comprising administering to the subject a death receptor agonist prior to, simultaneously with, or after administration of the compound of claim 1.

24. The method of claim 23, wherein the death receptor agonist is TRAIL or a TRAIL receptor antibody.
